(19) United States
(12) Patent Application Publication
(10) Pub. No.: US 2017/0035864 A1
(43) Pub. Date: Feb. 9, 2017

(54) SPECIFIC VIRUS-LIKE PARTICLE-CpG OLIGONUCLEOTIDE VACCINES AND USES THEREOF

(71) Applicant: Bullet Biotechnology, Inc., Redwood City, CA (US)

(72) Inventor: Thomas THERIAULT, Menlo Park, CA (US)

(21) Appl. No.: 15/103,300

(22) PCT Filed: Dec. 9, 2014
(86) PCT No.: PCT/US14/69406

§ 371 Date: Jun. 9, 2016

Related U.S. Application Data
(60) Provisional application No. 61/913,787, filed on Dec. 9, 2013.

(51) Int. Cl. A61K 39/00; A61K 45/06; C12N 7/00; C12N 15/117

(52) U.S. Cl. CPC A61K 39/0011; C12N 15/117; A61K 45/06; C12N 7/00; A61K 2039/55561; C12N 2310/351; C12N 2310/17; C12N 2320/30; A61K 2039/5258; C12N 2310/315; A61K 2039/585; C12N 2730/10134; C12N 2730/10123; C12N 2730/10122

(57) ABSTRACT

The invention provides vaccines containing, as its only active ingredient, a VLP having a CpG oligonucleotide attached thereto and a non-toxic pharmaceutically acceptable carrier or diluent and uses thereof. The invention further provides a pharmaceutical composition comprising a vaccine consisting of a VLP having a CpG oligonucleotide, one or more non-toxic pharmaceutically acceptable carrier or diluent, and a therapeutic agent admixture therewith and uses thereof.

Throughout this application various publications are referenced. The disclosures of these publications in their entirety are hereby incorporated by reference into this application in order to more fully describe the state of the art to which this invention pertains.

### BACKGROUND OF THE INVENTION

Cancer is a major health problem that needs better treatment regimens. The American Cancer Society estimates that in 2012 more than 1,638,910 people were newly diagnosed with cancer and 577,190 people died from cancer. Many cancers, such as triple-negative breast cancer, brain cancer, and pancreatic cancer, are still fatal diseases with no cure. Patients can face years of treatments that are difficult to tolerate and have many adverse events. Five-year survival rates for stage IV breast cancer patients are about 22%, glioblastoma patients range from 4% to 17%, and from 1% to 14% for pancreatic cancer patients.

Therapeutic vaccines have started demonstrating very good potential in cancer: sipuleucel-T is now an approved dendritic-cell vaccine for prostate cancer and historical Idiotype (Id) vaccine programs demonstrated that a specific anti-Id immune response correlates strongly with progression-free and overall survival. Unfortunately, previous vaccines did not consistently produce a strong immune response, and, for example, treatment with sipuleucel-T is a cumbersome process that is not effective in many patients.

The invention solves the problem of the art by providing novel specific virus-like particles (VLPs) attached or joined to CpG that will induce an immune response sufficient for use as a therapeutic agent against diseases such as cancer, e.g., a solid tumor cancer.

### SUMMARY OF THE INVENTION

The invention provides vaccines comprising, as its only active ingredients, a VLP attached or joined to CpG oligonucleotides and one or more non-toxic pharmaceutically acceptable carrier or diluent and uses thereof. Further, also provided are compositions comprising such vaccines and a therapeutic agent, admixed therewith, and uses thereof.

The invention further provides vaccines comprising, as its only active ingredients, a VLP attached or joined to a CpG oligonucleotide and one or more immune checkpoint protein inhibitors and one or more pharmaceutically acceptable carriers, binders, diluents, adjuvants, excipients, and/or vehicles and uses thereof.

### BRIEF DESCRIPTION OF THE FIGURES

FIG. 1. Amino acid and nucleotide sequences of a Hepatitis B core antigen (HBC).

FIG. 2. Examples of nucleotide sequences of CpG-X.

FIG. 3. Analysis of expression of Hep B Core using methionine replacement. Hep B Core plasmid-containing E. coli were expanded in shake flasks in minimal media prior to continued expansion in a 2 liter bioreactor with minimal methionine followed by induction with IPTG and addition of a 1:20 ratio of methionine to azidohomoalanine. Purified Hep B Core monomer, a molecular weight standard, pre- and post-expression induction samples are shown.

FIG. 4. Analysis of conjugation of CpG oligonucleotide to VLP. Reducing SDS-PAGE analysis of 3 conjugation reactions of VLP produced by E. Coli and CpG-X. Briefly, 1.25 mg VLP at 60 uM equivalent monomer, 80 uM CpG-X, 200 uM ascorbate, 250 uM TTMA enhancer, 500 uM Cu(I) were prepared and mixed in Argon sparged 10 mM potassium phosphate, 0.01% Tween-20, pH 8 in a total reaction volume of 1250 microliters. Reactions were allowed to proceed at 30 degrees overnight prior to analysis by SDS-PAGE.

FIG. 5 shows the DNA construct used to express a 149-amino acid HepB core protein for production of a VLP of the invention. FIG. 5-1 shows a diagram of pET21-HepB Core plasmid DNA with relevant biological signals, coding sequences and restriction enzyme cleavage sites. FIG. 5-2 to FIG. 54 shows complete nucleotide sequence of pET21-HepB Core construct along with the location of the Hep B core protein coding sequence (bold and underlined) and T7 promoter (underline).

### DETAILED DESCRIPTION OF THE INVENTION

Definitions

“Vaccine” as used herein, is a preparation comprising a virus-like particle (VLP) of the invention that when administered stimulates an immune response in a mammal suffering from a cancer. A therapeutic vaccine may be administered during or after onset of a cancer. A prophylactic treatment vaccine may be administered prior to onset of the disease such as a cancer and is intended to prevent, inhibit or delay onset of the disease.

“VLP” as used herein is a virus-like particle made from non-infectious subunits of a virus that form a structure, commonly in the form of an icosahedral matrix. VLP is permissive to multi-valent display of CpG oligonucleotides and/or immune checkpoint inhibitors. The VLP may contain an assemblage of capsid protein monomers/subunits, for example, about a multiple of 60 coat or capsid protein monomers/subunits. VLPs based on an icosahedral structure may be formed by coat protein subunits with, e.g., 60 (T=1), 120 (T=2), 180 (T=3), 240 (T=4), 360 (T=7d), 420 (T=7), 780 (T=13), 960 (T=16), 1260 (T=21), 1500 (T=25), or 1620 (T=27) capsid proteins among other icosahedrons with different number of coat proteins. In the case of VLP based on Hepatitis B virus (HBV), in one embodiment, 180 or 240 HBV coat protein monomers (also referred to herein as viral coat polypeptides) can form two different types of VLPs arranged with, e.g., T=3 or T=4 icosahedron symmetry, respectively. For VLPs formed from HBV core protein (also referred to as HepB core protein), the invention provides in one embodiment a HBV coat protein truncated at the C-terminus leaving intact the first 149 amino acid at the N-terminus (aa 1-149), and the HepB Core VLP is formed by the assembly of, e.g., 180 or 240 HepB core monomer proteins.

For example, VLP-azide refers to the presence of at least one azide functional group in VLP, such as through the incorporation of a non-natural amino acid with an azide functional group, e.g., azidohomoalanine. Azidohomoalanine may be used to substitute for methionine in a polypeptide chain in vivo by supplying azidohomoalanine to a methionine auxotroph grown in methionine-deficient medium. Alternatively, azidohomoalanine may be introduced in vitro synthesis using a cell-free protein synthesis (CFPS) system. Presence of an azide functional group permits participation in copper-catalyzed [3+2] cycloaddition or “click chemistry” with an alkyne function group. Other non-natural amino acids with an azide function group are available and may be introduced into a polypeptide including VLP monomer or capsid proteins. Such non-natural amino acids with an azide function group include p-azido-L-phenylalanine.

For example, VLP-alkyne refers to the presence of at least one alkyne functional group in VLP, such as through the incorporation of a non-natural amino acid with an alkyne functional group, e.g., by supplying homopropargylglycine as a partial or complete substitute for methionine while expressing VLP in methionine auxotroph strains thus replacing methionine with the alkyne-containing non-natural amino acid. Alternatively, a non-natural amino acid may also be incorporated into a polypeptide at a desired site through the introduction of stop codon, e.g., amber stop codon UAG, and use of a suppressor tRNA charged with the desired non-natural amino acid, e.g., p-propargyloxyphenylalanine, permitting site-specific incorporation of a non-natural amino acid through suppression of an engineered stop codon in a RNA transcript encoding a specific polypeptide (Bundy and Swartz, Bioconjugate Chem. 21:255-263 (2010)). In either case, presence of an alkyne functional group permits participation in copper-catalyzed [3+2] cycloaddition or “click chemistry” with an azide function group.

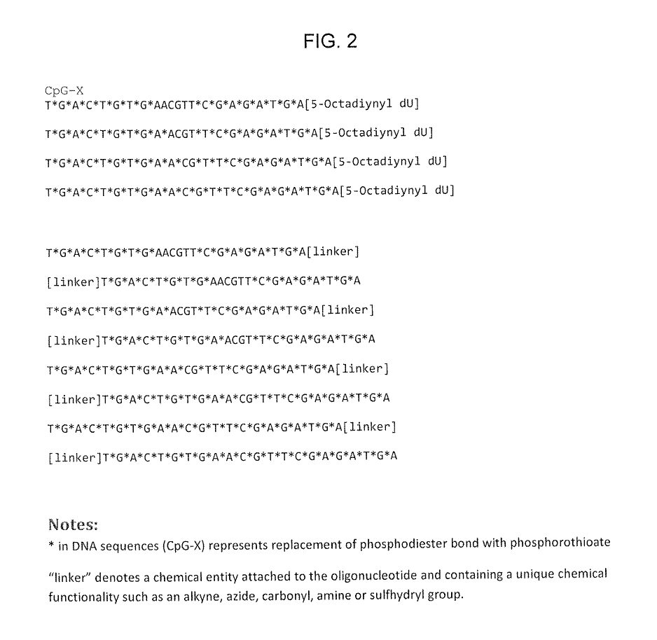

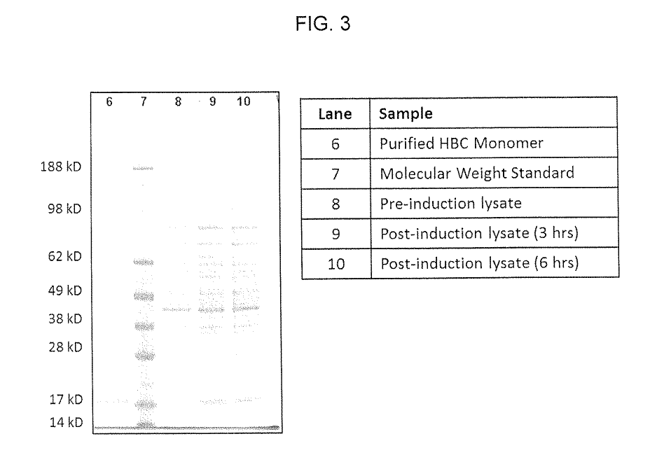

“CpG oligonucleotide” as used herein refers to an unmethylated oligonucleotide containing one or more cytosine-guanine dinucleotides joined by a phosphodiester or phosphorothioate backbone. The CpG oligonucleotide may have a phosphodiester, phosphorothioate or mixed phosphodiester-phosphorothioate backbone. These motifs may be recognized by mammals as “pathogen-associated molecular patterns” by Toll-Like Receptor 9. Examples of CpG oligonucleotides include, but are not limited to, the sequences shown in FIG. 2 (see also section on COMPOSITIONS OF THE INVENTION).

FIG. 2 also shows CpG oligonucleotides coupled to a linker such as 5-octadiynyl dU, which are designated as “CpG-X” wherein the “X” represents a crosslinking agent (a bifunctional crosslinking agent) or linker. The “linker” denotes a chemical entity attached to the oligonucleotide and contains a chemical functionality such as an alkyne, azide, carbonyl, amine or sulfhydryl group. Additional examples of linkers include, but are not limited to, maleimide, polyethylene glycol (PEG), bifunctional crosslinking agent, polyethylene glycol derivatives such as succinimide-maleimide PEG or SM(PEG)n with an NHS ester at one end and a maleimide group at the other, peptide linker, peptide nucleic acid linker (PNA), and modified nucleic acid linker.

As used herein “Immune checkpoint inhibitors” refers to agents that block immune checkpoints. Immune checkpoints are inhibitory pathways present in immune cells important for maintaining self-tolerance and controlling the degree of an immune response. Blocking these pathways may lead to reduced modulation of immune cells, or increased activation of immune cells.

The term “vector,” “construct” or “plasmid” as used herein refers to a recombinant nucleic acid molecule containing a desired coding sequence and appropriate nucleic acid sequences necessary for the expression of the coding sequence in a particular host organism. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A “vector,” “construct” or “plasmid” may also be used outside the context of a particular host organism, such as in a cell free protein synthesis system following production RNA transcripts or in an in vitro transcription-translation system.

As used herein, an “active ingredient” includes any compound or composition of matter which, when administered to an organism (human or animal subject) induces a desired pharmacologic and/or physiologic effect by local and/or systemic action.

Accordingly, the phrase “only active ingredient” in the context of an embodiment of the invention means that the VLPs attached to CpG oligonucleotides are the sole or exclusive active ingredients in the vaccine. However, the VLPs attached to CpG oligonucleotides are not the sole or exclusive ingredients in the vaccine since it contains excipients and other non-active agents necessary, e.g., for formulating the vaccine for storage or proper administration into a subject. Further, the invention provides pharmaceutical compositions or formulations that includes the vaccines and one or more therapeutic agents, e.g., admixed therewith.

As used herein, a “subject” means a mammal. The mammal can be a human or an animal such as a non-human primate, mouse, rat, dog, cat, horse, monkey, ape, rabbit or cow, but are not limited to these examples. Mammals, other than humans, can be advantageously used as subjects that represent animal models of disorders associated with, e.g., cancer. In addition, the methods and compositions described herein can be used to treat domesticated animals and/or pets. The terms, “patient” and “subject” are used interchangeably. A subject can be male or female.

The VLP vaccines of the invention may be administered in the form of a pharmaceutical composition comprising the active ingredient in a pharmaceutically acceptable dosage form. Depending upon the type of disease and patient to be treated, as well as the route of administration, the compositions may be administered at varying doses. Administration may be by methods including, but not limited to, intratumoral delivery, peritumoral delivery, intraperitoneal delivery, intrathecal delivery, intramuscular injection, subcutaneous injection, intravenous delivery, nasal spray and other mucosal delivery (e.g. transmucosal delivery), intra-arterial delivery, intraventricular delivery, intrasternal delivery, intracranial delivery, intradermal injection, electroincorporation (e.g., with electroporation), ultrasound, jet injector, and topical patches.

Formulations suitable for administration include aqueous and non-aqueous sterile injection solutions which may contain anti-oxidants, buffers, bacteriostats and solutes which render the formulation isotonic with the blood of the intended recipient, and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

When a VLP vaccine of the invention described herein is being given to a subject, a skilled artisan would understand that the dosage depends on several factors, including, but not limited to, the subject's weight, disease and progression thereof or tumor size or tumor progression. With respect to duration and frequency of treatment, it is typical for skilled clinicians to monitor subjects in order to determine whether the treatment is providing therapeutic benefit, and to determine whether to increase or decrease dosage, increase or decrease administration frequency, discontinue treatment, resume or make other alterations to the treatment regimen.

In an embodiment, a non-limiting example of an administration protocol useful for the invention comprises multiple administrations of the multivalent VLP vaccine of the invention during an initial period (such as, for example, a six week period, with, for example, administration every two weeks). Furthermore, an administration protocol may also include multiple administrations of the multivalent VLP vaccine of the invention at first administration (such as at multiple sites within a tumor at first administration of the multivalent VLP vaccine).

By “effective amount” as used herein with respect to a VLP vaccine of the invention, is meant an amount of the multivalent VLP, administered to a subject that results in an immune response by the mammal so as to inhibit the disease such as cancer. Further, an effective amount may include any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, “inhibiting a tumor” may be measured in any way as is known and accepted in the art, including complete regression of the tumor(s) (complete response); reduction in size or volume of the tumor(s) or even a slowing in a previously observed growth of a tumor(s), e.g., at least about a 10-30% decrease in the sum of the longest diameter (LD) of a tumor, taking as reference the baseline sum LD (partial response); mixed response (regression or stabilization of some tumors but not others); or no apparent growth or progression of tumor(s) or neither sufficient shrinkage to qualify for partial response nor sufficient increase to qualify for progressive disease, taking as reference the smallest sum LD since the treatment started (stable disease).

Tumor or cancer status may also be assessed by sampling for the number, concentration or density of tumor or cancer cells, alone or with respect to a reference. Tumor or cancer status may also be assessed through the use of surrogate marker(s), such as Her-2 in breast cancer or PSA in prostate cancer.

As used herein, “treating” means using a therapy to ameliorate a disease or disorder or one or more of the biological manifestations of the disease or disorder; to directly or indirectly interfere with (a) one or more points in the biological cascade that leads to, or is responsible for, the disease or disorder or (b) one or more of the biological manifestations of the disease or disorder; to alleviate one or more of the symptoms, effects or side effects associated with the disease or disorder or one or more of the symptoms or disorder or treatment thereof; or to slow the progression of the disease or disorder or one or more of the biological manifestations of the disease or disorder. Treatment includes eliciting a clinically significant response. Treatment may also include improving quality of life for a subject afflicted with the disease or disorder (e.g., a subject afflicted with a cancer may receive a lower dose of an anti-cancer drug that cause side-effects when the subject is immunized with a composition of the invention described herein). Throughout the specification, compositions of the invention and methods for the use thereof are provided and are chosen to provide suitable treatment for subjects in need thereof.

In some embodiments, treatment with a composition of the invention described herein induces and/or sustains an immune response in a subject. Immune responses include innate immune response, adaptive immune response, or both. Innate immune response may be mediated by neutrophils, macrophages, natural killer cells (NK cells), and/or dendritic cells. Adaptive immune response includes humoral responses (i.e., the production of antibodies), cellular responses (i.e., proliferation and stimulation of T-lymphocytes), or both. Measurement of activation and duration of cellular response may be by any known methods including, for example, cytotoxic T-lymphocyte (CTL) assays. Humoral responses may be also measured by known methods including isolation and quantitation of antibody titers specific to the compositions of the invention (e.g., vaccines) such as IgG or IgM antibody fractions.

In some embodiments, the methods of treatment (e.g., immunotherapy) described herein is used as a stand-alone therapy without combining with any other therapy.

In other embodiments, the methods of treatment (e.g., immunotherapy) described herein provide adjunct therapy to other therapies, e.g., cancer therapy, prescribed for a subject. For example, the methods of treatment (e.g., immunotherapy) described herein may be administered in combination with radiotherapy, chemotherapy, gene therapy or surgery. The combination is such that the method of treatment (e.g., immunotherapy) described herein may be administered prior to, with or following adjunct therapy.

In accordance with the invention, the effect of anti-disease or disorder treatment (e.g., a cancer treatment) may be assessed by monitoring the patient, e.g., by measuring and comparing survival time or time to disease progression (disease-free survival). Any assessment of response may be compared to individuals who did not receive the treatment or were treated with a placebo, or to individuals who received an alternative treatment.

As used herein, “preventing” is understood to refer to the prophylactic administration of a drug to substantially diminish the likelihood or severity of a condition or biological manifestation thereof, or to delay the onset of such condition or biological manifestation. One skilled in the art will appreciate that prevention is not an absolute term. Prophylactic therapy is appropriate, for example, when a subject is considered at high risk for developing a particular disease or disorder (e.g., cancer), such as when a subject has a strong family history of a disease or disorder or when a subject has been exposed to e.g., a disease causing agent, e.g., a carcinogen.

Other than in the operating examples, or where otherwise indicated, all numbers expressing quantities of ingredients or reaction conditions used herein should be understood as modified in all instances by the term “about”. The term “about” when used in connection with percentages can mean a range of ±1-10%.

The use of the singular includes the plural unless specifically stated otherwise. The word “a” or “an” means “at least one” unless specifically stated otherwise. The use of “or” means “and/or” unless stated otherwise. The meaning of the phrase “at least one” is equivalent to the meaning of the phrase “one or more.” Furthermore, the use of the term “including,” as well as other forms, such as “includes” and “included,” is not limiting. The use of the term “containing,” as well as other forms, such as “contains” and “contained,” is not limiting. Also, terms such as “element” or “component” encompass both elements or components comprising one unit and elements or components comprising more than one unit unless specifically stated otherwise.

Compositions of the Invention

The invention provides a vaccine comprising or containing a (1) VLP having a CpG oligonucleotide attached thereto and (2) one or more non-toxic pharmaceutically acceptable carriers or diluents. One or more copies of CpG oligonucleotide may be attached or joined to the surface of the VLP. In one embodiment, the only active ingredients in the vaccine are the VLPs containing one or more copies of CpG oligonucleotides attached thereto. The CpG oligonucleotide may be an agonist of a TLR9 molecule.

The invention further provides a vaccine containing or consisting essentially of (1) a VLP having one or more CpG oligonucleotides and immune checkpoint inhibitors attached thereto and (2) one or more non-toxic pharmaceutically acceptable carriers or diluents. Each of the CpG oligonucleotides and immune checkpoint inhibitors may be attached or joined to the surface of the VLP. In one embodiment, the only active ingredients in the vaccine are the VLPs containing one or more copies of CpG oligonucleotides and immune checkpoint inhibitors attached thereto.

Further, the VLP free of a viral genome may comprise virus coat polypeptides derived from any of an Adenoviridae, Picornaviridae, Herpesviridae, Hepadnaviridae, Flaviviridae, Retroviridae, Orthomyxoviridae, Paramyxoviridae, Papillomaviridae, Rhabdoviridae, Togaviridae or Paroviridae families. Preferably, the VLP is a stable icosahedral VLP free of a viral genome.

Specifically, examples of viruses from which the virus coat proteins may be derived include but are not limited to any of a bacteriophage, adenovirus, coxsackievirus, Hepatitis A virus, poliovirus, Rhinovirus, Herpes simplex virus, Varicella-zoster virus, Epstein-Barr virus, Human cytomegalovirus, Human herpes virus, Hepatitis B virus, Hepatitis C virus, yellow fever virus, dengue virus, West Nile virus, HIV, Influenza virus, Measles virus, Mumps virus, Parainfluenza virus, Respiratory syncytial virus, Human metapneumovirus, Human papillomavirus, Rabies virus, Rubella virus, Human bocavirus or Parvovirus, and Norovirus. In one embodiment, the bacteriophage may be a M52 bacteriophage, P1 like viruses, P2 like viruses, T4 like viruses, P22 like viruses, and lambda-like viruses. A VLP derived from Hepatitis B virus is preferred.

In an embodiment of the invention, the average amount of CpG oligonucleotide attached to VLP may be an equivalent to 1 to 10 copies of CpG oligonucleotide per VLP, 10 to 50 copies of CpG oligonucleotide per VLP, 40 to 80 copies of CpG oligonucleotide per VLP, 70 to 170 copies of CpG oligonucleotide per VLP or 160 to 240 copies of CpG oligonucleotide per VLP. In another embodiment, the CpG oligonucleotide attached to VLP protein monomers may be in an amount such that the CpG oligonucleotide to VLP weight ratio is equivalent to 1:1000 to 1:100, 1:100 to 1:10, 1:10 to 1:4, 1:4 to 1:2 or 1:2 to 1:1. In yet another embodiment, the CpG oligonucleotide attached to a VLP protein monomer is in an amount (molar) such that the CpG oligonucleotide to VLP monomer ratio is equivalent to 1:24 to 1:12, 1:12 to 1:6, 1:6 to 1:3, 1:3 to 2:3 or 1:2 to 1:1.

In an embodiment of the invention, the CpG oligonucleotide comprises a sequence, 5′ TGACTGTGAACGTTCGAGATGA-3′. The nucleic acid molecule, oligonucleotide or CpG oligonucleotide may be a modified oligonucleotide with a mixture of phosphodiester and phosphorothioate bonds in the sequence, 5′ T*G*A*C*T*G*T*G*AACGTT*C*G*A*G*A*T*G*A 3′ or 5′ T*G*A*C*T*G*T*G*A*ACGT*T*C*G*A*G*A*T*G*A 3′ or 5′ T*G*A*C*T*G*T*G*A*A*CG*T*T*C*G*A*G*A*T*G*A 3′, Or 5′ T*G*A*C*T*G*T*G*A*A*C*G*T*T*C*G*A*G*A*T*G*A 3′, where * represents replacement of a phosphodiester bond with a phosphorothioate bond. Still other embodiments of the CpG oligonucleotide incorporate a linker into the molecule (CpG-X), for example, by coupling a chemical entity containing a unique chemical functionality such as an alkyne, azide, carbonyl, amine or sulfhydryl group to either the 5′ end or 3′ end of the sequence, for example, {linker}-5′ T*G*A*C*T*G*T*G*A*A*CG*T*T*C*G*A*G*A*T*G*A 3′ or 5′ T*G*A*C*T*G*T*G*A*A*CG*T*T*C*G*A*G*A*T*G*A-{linker} 3′, respectively. In a preferred CpG-X embodiment, an alkyne functional group is introduced into the CpG molecule, for example, by coupling 5-octadiynyl dU {5-Oct-dU} to the 3′ end of the sequence, for example, 5′ T*G*A*C*T*G*T*G*A*A*CG*T*T*C*G*A*G*A*T*G*A-{5-Oct-dU} 3′ or 5′ T*G*A*C*T*G*T*G*A*A*C*G*T*T*C*G*A*G*A*T*G*A-{5-Oct-dU} 3′. The alkyne functional group may participate in a (3+2) cycloaddition click reaction with an azide functional group incorporated into a capsid protein of a VLP, resulting in VLP crosslinked to a CpG oligonucleotide.

Additional examples of CpG oligonucleotides include, but are not limited to, (1) 5′ TCG TCG TTG TCG AAC GTT CGA GAT GAT 3′ (designated M353); (2) 5′ TCG TCG TTC GAA CGA GAT GAT 3′ (designated M355); (3) 5′ TCG TCG TTT TGT CGA ACG TCC GAG ATG AT 3′ (designated M354); (4) 5′ TCG TCG AAC GTT CGA GAT GAT 3′ (designated M352); (5) 5′ TCG TCG AGC GCT CGA GAT GAT 3′ (designated C593); (6) 5′ TCG TCG ATC GAT CGA GAT GAT 3′ (designated C594); (7) 5′ TCG TCG GTC GAC CGA GAT GAT 3′(designated C595); (8) 5′ TCG TCG TTC GAA CGA GAT GAT 3′ (designated C546); (9) 5′ TCG TCG GGC GCC CGA GAT GAT 3′ (designated C640); (10) 5′ TCG TCG CGC GCG CGA GAT GAT 3′ (designated C642); (11) 5′ TCG TCG CCC GGG CGA GAT GAT 3′ (designated C644); (12) 5′ TCG TCG ACG ATC GTC GAT GAT 3′ (designated M356); (13) 5′ TCG TCG TCG TAC GAC GAT GAT 3′ (designated M357); (14) 5′ TCG TCG TTG TCG TTC GAA CGA CGT TGA T 3′ (designated M361); (15) 5′ TCG TCG TCG TTC GAA CGA CGT TGA T 3′(designated M362); (16) 5′ TCG TCG TTC TCG ACG ATC GTC GAT GAT 3′(designated M358); (17) 5′ TGA CTG TGA ACG TTC GGA TGA 3′ (designated 1018); (18) 5′ TCG TCG AAC GTT CGA GAT GAT 3′ (designated C274); (19) 5′ TCG AAC GTT CGA ACG TTC GAA T 3′ (designated C583); (20) 5′ TTC GAA CGT TCG AAC GTT CGA AT 3′ (designated C582); (21) 5′ TCA ACG TTC GAA CGT TCG AAC GTT 3′ (designated C637); (22) 5′ TCG TCG TTT TGT CGT TTT GTC GTT 3′ (designated 2006); (23) 5′ TCG ACG TCG ACG TCC ACG TAT 3′ (designated C630); (24) 5′ TCG TCG AAA CGT TTC GAC AOT 3′ (designated C631); (25) 5′ TCG TCG AAA ACG TTT TCG AGA T 3′ (designated C633); (26) 5′GGg gga cga tcg tcg GGG Gg 3′(designated 2216[12]); (27) 5′ TCG TCG TTT TGT CGT TTT GTC GTT 3′ (designated 2006[5]); (28) 5′ TGC TGC TGC TTG CAA GCA OCT TGA T 3′ (designated M383); (29) 5′ TCG TCG TCG TTC GAA CGA CGT TGA T 3′ (designated M384); (30) 5′ TCG TCG TGC TTG CAA GCA CGT TGA T 3′ (designated M385); (31) 5′ TCG TCG TCG ATC GTA CGA CGT TGA T 3′ (designated M386); (32) 5′ TCG TCG TCG TTC GAA CGA CO 3′ (designated M387); and (33) 5′ TCG TCG TCG TTC GAA CGA CGT CGT T 3′ (designated M388) (Marshall et al., Journal of Leukocyte Biology, 2003, 73: 781-792; Hartmann, G. et al., Eur. J. Immunol. 2003, 33:1633-41). The CpG oligonucleotides above may have a phosphodiester, phosphorothioate or mixed phosphodiester-phosphorothioate backbone.

For attachment of the CpG oligonucleotide to the VLP, the virus coat polypeptides of the VLP may be modified to comprise at least one first unnatural amino acid (also referred to herein as non-natural amino acid or non-canonical amino acid (nnAA)) at a site of interest, such as the incorporation of azidohomoalanine during virus coat polypeptide synthesis in the place of methionine, and the CpG oligonucleotide attached to an alkyne functional group, such as 5-octadiynyl dU at the 3′ end of the CpG oligonucleotide to produce CpG-X. The azide functional group of azidohomoalanine incorporated into a capsid protein of a VLP may participate in a (3+2) cycloaddition click reaction with an alkyne functional group of CpG-X, resulting in VLP crosslinked to CpG oligonucleotide. Other unnatural amino acid-containing capsid proteins within the same VLP may similarly participate in the (3+2) cycloaddition click reaction to produce a VLP attached or joined to a CpG oligonucleotide, producing a VLP with two or more CpG oligonucleotides.

Similarly, in the embodiments where the vaccine contains VLPs also having immune checkpoint inhibitors attached thereto, the immune checkpoint inhibitors may be modified to comprise at least one second unnatural amino acid, wherein the first unnatural amino acid is different from, and reactive with the second unnatural amino acid. An example of one first unnatural amino acid is azidohomoalanine. An example of a second unnatural amino acid is propargyloxyphenylalanine. The azide functional group of azidohomoalanine incorporated into a capsid protein of a VLP may participate in a (3+2) cycloaddition click reaction with an alkyne functional group of propargyloxyphenylalanine incorporated into a polypeptide agent, such as a polypeptide-based immune checkpoint inhibitor, resulting in VLP crosslinked to a polypeptide agent. Other unnatural amino acid-containing capsid proteins within the same VLP may similarly participate in the (3+2) cycloaddition click reaction to produce a VLP attached or joined to an immune check point inhibitors, producing A VLP with two or more immune check point inhibitors.

Using a similar strategy, VLP with reactive azide functional groups could be coupled to other non-proteinaceous or non-nucleic acid-based therapeutic agents, such as antagonist ligands or inhibitors, including small molecule inhibitors, of immune checkpoint proteins which are not protein or nucleic acid, through functionalizing these agents with an alkyne functional group. Such non-proteinaceous or non-nucleic acid-based agents may be attached to a VLP through the (3+2) cycloaddition click reaction to produce a VLP attached or joined to non-proteinaceous or non-nucleic acid-based agents.

In an embodiment of the invention, the VLP contains at least one or at least two unnatural amino acid per capsid monomer subunit. For example, at least one-fiftieth of the total number of unnatural amino acids in a VLP may be used to attach a CpG oligonucleotide. In another embodiment, one-twentieth of the total number of unnatural amino acids in a VLP may be used to attach a CpG oligonucleotide. In another embodiment, one-tenth of the total number of unnatural amino acids in a VLP may be used to attach a CpG oligonucleotide. In another embodiment, about one fourth of the total number of unnatural amino acids in a VLP may be used to attach a CpG oligonucleotide. In a further embodiment, about one-third of the total number of unnatural amino acids in a VLP may be used to attach a CpG oligonucleotide. In yet another embodiment, about one half of the total number of unnatural amino acids in a VLP may be used to attach a CpG oligonucleotide. For example, about two-thirds of the total number of unnatural amino acids in a VLP may be used to attach a CpG-X oligonucleotide. In another example, about four-fifths of the total number of unnatural amino acids in a VLP may be used to attach a CpG-X oligonucleotide.

Also, in an embodiment of the invention, in the VLP, at least one-twenty fifth of the viral coat proteins may display a CpG oligonucleotide attached thereto. In another embodiment, at least one-tenth of the viral coat proteins may display a CpG oligonucleotide. In another embodiment, at least one-fifth of the viral coat proteins may display a CpG oligonucleotide. In yet another embodiment, about half of the viral coat proteins may display a CpG oligonucleotide. In a further embodiment, about two-thirds of the viral coat proteins may display a CpG oligonucleotide. In yet another embodiment, nearly all of the viral coat proteins may display a CpG oligonucleotide.

In another embodiment, the VLP free of a viral genome of the invention further comprises a CpG oligonucleotide with an initially reactive linker at the 3′ or 5′ end and used to couple (e.g., covalently attached) to the VLP or VLP capsid protein, e.g., an initially reactive linker which participates in a chemical reaction so as to crosslink the CpG oligonucleotide to the VLP or VLP capsid protein. Examples of linkers (such as reactive linkers) include a chemical functionality such as an alkyne, azide, carbonyl, amine or sulfhydryl group. In a preferred embodiment of the invention, the reactive linker is a 5-octadiynyl deoxyuridine, a modified deoxyuridine which is located at the 3′ end of a CpG oligonucleotide (e.g., as shown in FIG. 2). Following coupling to the VLP, the resultant product may be, e.g., a VLP covalently attached to a CpG oligonucleotide through a linker, but no longer with the reactive functional group which participated in the covalent bond formation reaction.

Further, the invention additionally provides for a pharmaceutical composition for treatment of a solid tumor cancer comprising any of the VLP vaccine of the invention and one or more therapeutic agents that are admixed with the vaccine. In an embodiment where a second therapeutic agent is added, the second therapeutic agent may be the same as the therapeutic agent admixed with the vaccine or a different therapeutic agent.

Examples of therapeutic agents so admixed include, but are not limited to, an agent that inhibits an immune checkpoint protein (also referred to herein as an immune checkpoint inhibitor). Examples of immune checkpoint inhibitors include agents that inhibit PD-1 (e.g., a PD-1 inhibitor or an anti-PD-1 agent); CTLA-4 (e.g., a CTLA-4 inhibitor or an anti-CTLA-4 agent); LAG3 (e.g., a LAG3 inhibitor or an anti-LAG3 agent); KIR (e.g., a KIR inhibitor or an anti-KIR agent); TIM3 (e.g., an TIM3 inhibitor or an anti-TIM3 agent); TIGIT (e.g., a TIGIT inhibitor or an anti-TIGIT agent); BTLA (e.g., a BTLA inhibitor or an anti-BTLA agent); CD160 (e.g., a CD160 inhibitor or an anti-CD160 agent); VISTA (e.g. an VISTA inhibitor or an anti-VISTA agent); and A2aR (e.g., an A2aR inhibitor or an anti-A2aR agent). Alternatively, the immune checkpoint inhibitor may inhibit a ligand of a checkpoint receptor, examples of which would include PDL1 (e.g., a PDL1 inhibitor or an anti-PDL1 agent), PDL2 (e.g., a PDL2 inhibitor or an anti-PDL2 agent), B7-H3 (e.g., a B7-H3 inhibitor or an anti-B7H3 agent); B7-H4 (e.g., a B7-H4 inhibitor or an anti-B7-H4 agent). The agent may be an isolated antibody or fragment or derivative thereof that blocks the target receptor (e.g., PD-1, B7-H3, B7-H4, CTLA-4, LAG3, KIR, TIM3, TIGIT, BTLA, CD160, or A2aR) or a ligand. Further, the agent may be a small molecule that blocks activity of an immune checkpoint protein or a ligand. The ligand may be an antagonist or selective modulator of an immune checkpoint protein, such as a target receptor in an immune checkpoint pathway.

In accordance with the invention, the isolated antibody may be an isolated or purified monoclonal antibody. In further embodiments, the antibody or antigen-binding fragment is a labeled antibody, a bivalent antibody, a polyclonal antibody, a bispecific antibody, a chimeric antibody, a recombinant antibody, an anti-idiotypic antibody, a humanized antibody, or an affinity matured antibody. In other embodiments, the antigen-binding fragment is a camelized single domain antibody, a diabody, an scfv, an scfv dimer, a dsfv, a (dsfv)₂, a dsFv-dsfv′, a bispecific ds diabody, a Fv, a Fab, a Fab′, a F(ab′)₂, or a domain antibody. In other embodiments, the antigen-binding fragment is operably attached to a constant region, wherein the constant region is a kappa light chain, gamma-1 heavy chain, gamma-2 heavy chain, gamma-3 heavy chain or gamma-4 heavy chain.

In a further embodiment, the isolated antibody or antigen-binding fragment thereof may be conjugated to a therapeutic agent (e.g., a chemotherapeutic agent), a toxin, a radioisotope, or a detectable label.

Additional examples of therapeutic agents so admixed include, but are not limited to, an agent that is a co-stimulatory molecule. Examples of co-stimulatory agents include HVEM; ICOSL; 4-1BBL; OX40L; GITRL; CD40L; and agents that stimulate CD28 (e.g., a CD28 agonist); ICOS (e.g., an ICOS agonist); CD137 (e.g., a CD137 agonist); OX40 (e.g., an OX40 agonist); CD27 (e.g., an CD27 agonist); CD40 (e.g., a CD40 agonist); CD40L (also known as gp-39) (e.g., an CD40L agonist); LIGHT (e.g., a LIGHT agonist); LT-alpha (e.g., an LT-alpha agonist); GITR (e.g., a GITR agonist); and a mimic of a ligand of the aforementioned. The agent may be an isolated antibody or fragment or derivative thereof that stimulates the target receptor (e.g., CD28, ICOS, CD137, OX40, CD27, CD40, CD40L, LIGHT, LT-alpha, and/or GITR also known as TNFRSF18) such as an anti-CD28 antibody, anti-ICOS antibody, anti-CD137 antibody, anti-OX40 antibody, anti-CD27 antibody, anti-CD40 antibody, anti-CD40L antibody, anti-LIGHT antibody, anti-LT-alpha antibody, and anti-GITR antibody. Further, the agent may be a small molecule that stimulates the target receptor.

Yet another example of a therapeutic agent includes but is not limited to an agent that suppresses Treg activity. An example of a Treg suppressor includes agents that stimulate GITR (e.g., a GITR agonist), or a ligand, or a mimic of a ligand thereof. The agent may be an isolated antibody or fragment or derivative thereof that stimulates the target receptor (e.g., GITR). An example antibody is TRX-518. Another example protein is GITR-L. Further, the agent may be a small molecule that stimulates the target receptor.

Yet another example of a therapeutic agent includes but is not limited to an agent that depletes Treg cells. Examples of Treg depleting agents include agents that induce cell death in Treg cells (e.g., binding to a surface antigen on Treg cells (e.g., FR4, CD4, CD25 (IL-2Ra), CD127 (IL7Ra), CD45RA, CD45RO, CD39, CD73, GITR, CD101, GARP)) causing ADCC cytotoxicity (e.g., antibodies that mediate ADCC (antibody-dependent cell-mediated cytotoxicity)), CDC (complement-dependent cytotoxicity), or mediate cell death through other effector functions. Alternatively, examples of Treg depleting agents include agents that induce PCD (programmed cell-death). The agent may be an antibody or fragment or derivative thereof that induces cell death. Further, the agent may be a small molecule that induces cell death.

Yet another example of a therapeutic agent includes but is not limited to an agent (such as an antibody or small molecule) that binds to a tumor necrosis factor superfamily receptor (TNFRSFR) or ligand (TNFRSFRL). Examples include agents (such as an antibody or fragment or derivative thereof or small molecule) that stimulate a TNFRSFR or a ligand (e.g., CD137 agonist, an NGFR agonist, a BAFFR agonist, an Osteoprotegerin agonist, a BCMA agonist, a OX40 agonist, a CD27 agonist, a RANK agonist, a CD30 agonist, a RELT agonist, a CD40 agonist, a TACI agonist, a DcR3 agonist, a TNF RI agonist, a DcTRAIL R1 agonist, a TNF agonist, a DcTRAIL R2 agonist, a TRAIL R1 agonist, a DR3 agonist, a TRAIL R2 agonist, a DR6 agonist, a TRAIL R3 agonist, a EDAR agonist, a TRAIL R4 agonist, a Fas agonist, a TROY agonist, a GITR agonist, a TWEAK R agonist, a HVEM agonist, a XEDAR agonist, a Lymphotoxin beta receptor agonist, a 4-1BB agonist, a APRIL agonist, a BAFF agonist, a TL1A agonist, a TWEAK agonist, and a LIGHT agonist).

Examples also include inhibitors of a TNFRSFR or ligand thereof (e.g., CD137 antagonist, an NGFR antagonist, a BAFFR antagonist, an Osteoprotegerin antagonist, a BCMA antagonist, an OX40 antagonist, a CD27 antagonist, a RANK antagonist, a CD30 antagonist, a RELT antagonist, a CD40 antagonist, a TACI antagonist, a DcR3 antagonist, a TNF RI antagonist, a DcTRAIL R1 antagonist, a TNF antagonist, a DcTRAIL R2 antagonist, a TRAIL R1 antagonist, a DR3 antagonist, a TRAIL R2 antagonist, a DR6 antagonist, a TRAIL R3 antagonist, a EDAR antagonist, a TRAIL R4 antagonist, a Fas antagonist, a TROY antagonist, a GITR antagonist, a TWEAK R antagonist, a HVEM antagonist, a XEDAR antagonist, a Lymphotoxin beta receptor antagonist, a 4-1BB antagonist, a APRIL antagonist, a BAFF antagonist, a TL1A antagonist, a TWEAK antagonist, and a LIGHT antagonist). Merely by way of example, these inhibitors may be an antibody or fragment or derivative thereof or a small molecule directed against a TNFRSFR or a ligand thereof.

The therapeutic agent may be an anti-cancer agent that inhibits cell proliferation or induces apoptosis. Examples of therapeutic agents include, but are not limited to, lenalidomide; ipilimumab; rituximab; alemtuzumab; ofatumumab; flavopiridol; Adriamycin; Dactinomycin; Bleomycin; Vinblastine; Cisplatin; ABT-199; acivicin; aclarubicin; acodazole hydrochloride; acronine; adozelesin; aldesleukin; altretamine; ambomycin; ametantrone acetate; amino glutethimide; amsacrine; anastrozole; anthramycin; asparaginase; asperlin; azacitidine; azetepa; azotomycin; batimastat; benzodepa; bicalutamide; bisantrene hydrochloride; bizelesin; bleomycin sulfate; brequinar sodium; bropirimine; busulfan; cactinomycin; calusterone; caracemide; carbetimer; carboplatin; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; ibrutinib; idelalisib; idarubicin hydrochloride; ifosfamide; ilmofosine; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; obinutuzumab; ormaplatin; oxisuran; pegaspargase; peliomycin; pentamustine; peplomycin sulfate; perfosfamide; pipobroman; piposulfan; piroxantrone hydrochloride; plicamycin; plomestane; porfmer sodium; porfiromycin; prednimustine; procarbazine hydrochloride; puromycin; puromycin hydrochloride; pyrazofurin; riboprine; rituximab; rogletimide; safingol; safingol hydrochloride; semustine; simtrazene; sparfosate sodium; sparsomycin; spirogerranium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfm; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

In another embodiment, the therapeutic agent may be an alkylating agent which may be nitrogen mustards, ethylenimine and methylmelamines, alkyl sulfonates, nitrosoureas, or triazenes.

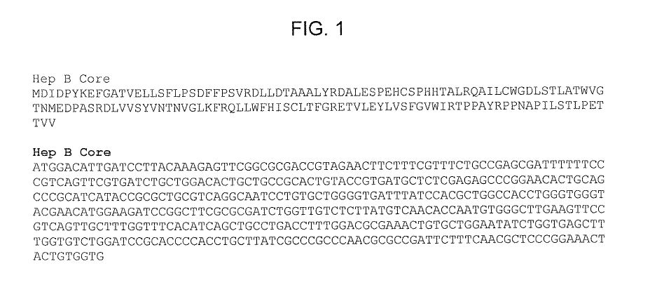

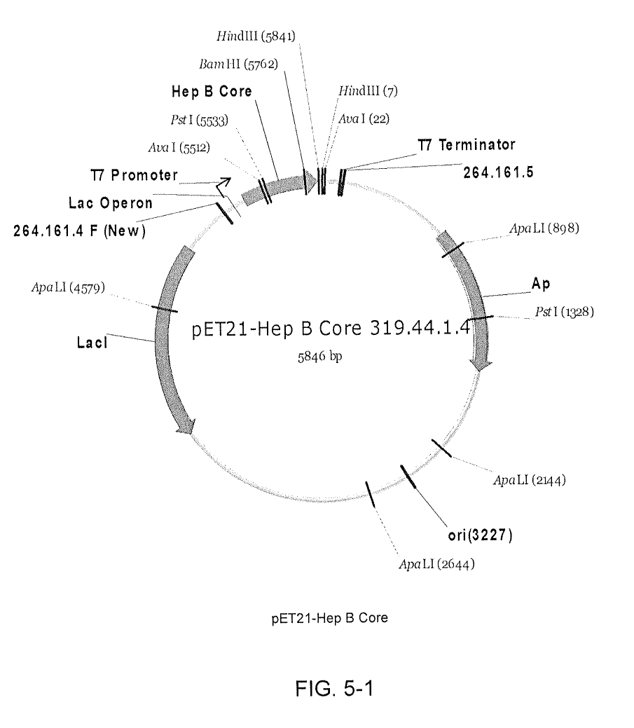

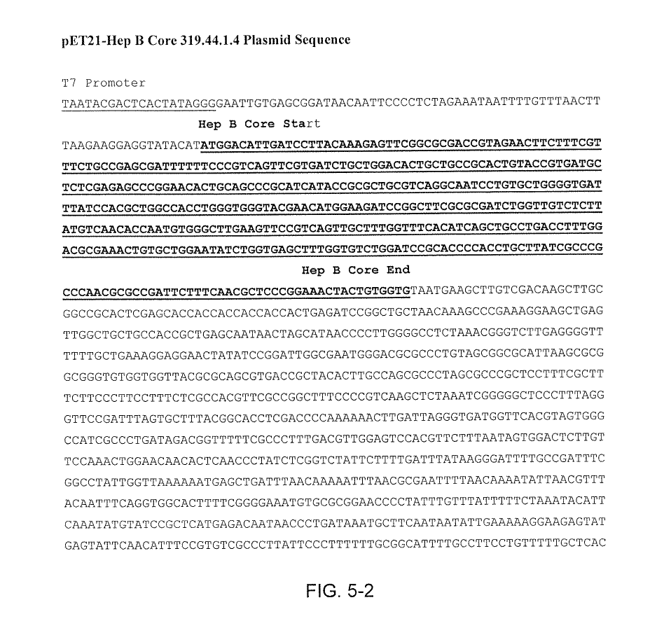

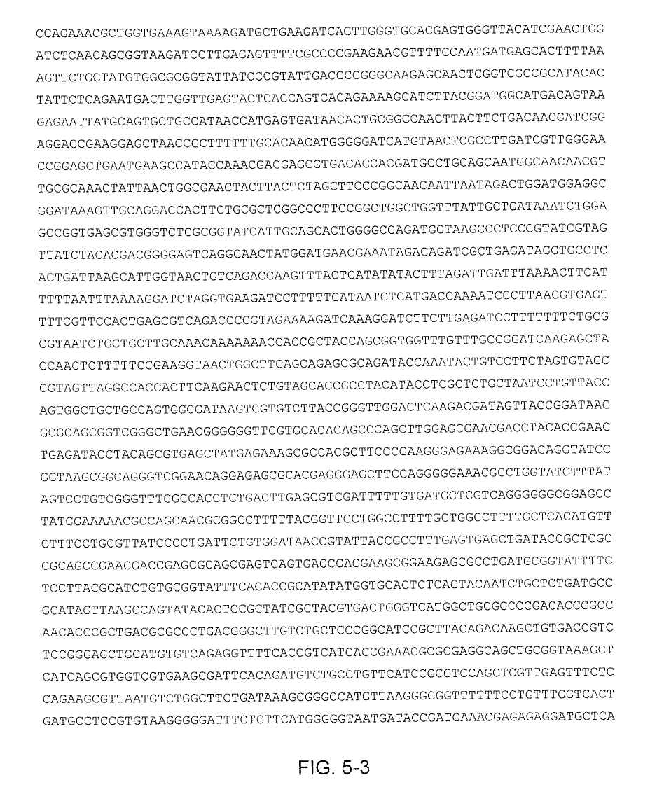

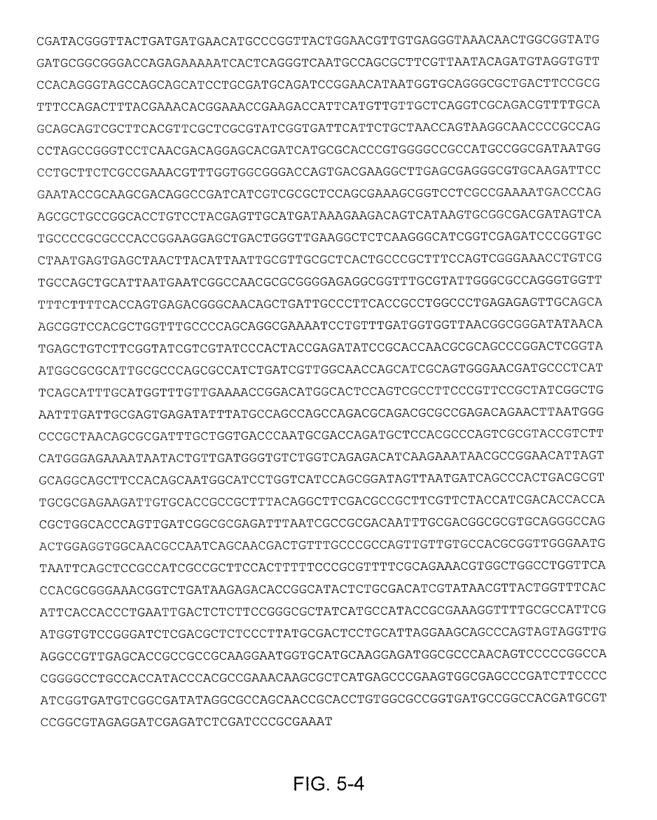

The invention further provides a nucleic acid molecule encoding the VLP of the invention, e.g., as shown in FIG. 1 or 5.

The nucleic acids of the invention may comprise nucleotide sequences and encode polypeptides (amino acid sequences) which are at least about 70% identical, preferably at least about 80% identical, more preferably at least about 90% identical and most preferably at least about 95% identical (e.g., 95%, 96%, 97%, 98%, 99%, 100%) to the reference nucleotide and amino acid sequences of the present invention (i.e., see examples herein, e.g., the sequences in FIGS. 1 and 2) when the comparison is performed by a BLAST algorithm wherein the parameters of the algorithm are selected to give the largest match between the respective sequences over the entire length of the respective reference sequences. Polypeptides comprising amino acid sequences which are at least about 70% similar, preferably at least about 80% similar, more preferably at least about 90% similar and most preferably at least about 95% similar (e.g., 95%, 96%, 97%, 98%, 99%, 100%) to the reference amino acid sequences of the present invention when the comparison is performed with a BLAST algorithm wherein the parameters of the algorithm are selected to give the largest match between the respective sequences over the entire length of the respective reference sequences, are also included in the present invention.

The nucleic acid molecule may be a DNA molecule (e.g., an isolated cDNA) encoding the VLP of the invention. Additionally, the nucleic acid molecule may be a RNA (e.g., an isolated RNA such as isolated mRNA). Alternatively, the nucleic acid molecule may be a hybrid of cDNA and mRNA. For example, the invention provides for a DNA construct comprising a vector that expresses the VLP free of a viral genome of the invention (see e.g., FIG. 5).

The nucleic acid molecules of the invention also include derivative nucleic acid molecules which differ from DNA or RNA molecules. Derivative molecules include peptide nucleic acids (PNAs), and non-nucleic acid molecules including phosphorothioate, phosphotriester, phosphoramidate, and methylphosphonate molecules, that bind to single-stranded DNA or RNA in a base pair-dependent manner (Zamecnik, P. C., et al., 1978 Proc. Natl. Acad. Sci. 75:280284; Goodchild, P. C., et al., 1986 Proc. Natl. Acad. Sci. 83:4143-4146). Reviews of methods for synthesis of DNA, RNA, and their analogues can be found, e.g., in: Oligonucleotides and Analogues, eds. F. Eckstein, 1991, IRL Press, New York; Oligonucleotide Synthesis, ed. M. J. Gait, 1984, IRL Press, Oxford, England.

Additionally, the invention provides a vector which comprises the nucleic acid molecule of the invention. The term vector includes, but is not limited to, plasmids, cosmids, and phagemids. The host vector system comprises the vector of the invention in a suitable host cell. Examples of suitable host cells include but are not limited to bacterial cell and eukaryotic cells.

In another embodiment, the invention provides a process comprising recovering a VLP of the invention and/or VLP monomers from a culture medium and from cultured cells. In the case of VLP monomers from a culture medium or cultured cells, such monomers may be first isolated and then allowed to form VLPs.

According to embodiments of the invention, the degeneracy of the genetic code provides a predictable number of nucleic acid sequences encoding the VLP of the invention, the codons of which may be selected to optimally express the isolated nucleic acid in a host organism (including without limitation, bacteria, yeast, mammalian cells cultured in vitro, and cells of a mammal (including a human). Such expression is useful for production of the nucleic acid or the polypeptide in a host organism for subsequent isolation and use according to the invention or in cell free in vitro transcription and/or translation system.

The terms “pharmaceutical formulations”, “pharmaceutical compositions” and “dosage forms” are used interchangeably herein and refer to a composition containing the active ingredient(s) of the invention in a form suitable for administration to a subject.

The pharmaceutical compositions of the present invention may be mixed with one or more pharmaceutically acceptable carriers, binders, diluents, adjuvants, excipients, or vehicles, such as preserving agents, fillers, polymers, disintegrating agents, glidants, wetting agents, emulsifying agents, suspending agents, lubricating agents, acidifying agents, dyes, preservatives and dispensing agents, or compounds of a similar nature depending on the nature of the mode of administration and dosage forms. Such ingredients, including pharmaceutically acceptable carriers and excipients that may be used to formulate dosage forms, are described in the Handbook of Pharmaceutical Excipients, American Pharmaceutical Association (1986), incorporated herein by reference in its entirety.

Pharmaceutically acceptable carriers are generally non-toxic to recipients at the dosages and concentrations employed and are compatible with other ingredients of the formulation. Examples of pharmaceutically acceptable carriers include water, saline, Ringer's solution, dextrose solution, ethanol, polyols, vegetable oils, fats, ethyl oleate, liposomes, waxes polymers, including gel forming and non-gel forming polymers, and suitable mixtures thereof. The carrier may contain minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulin; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG. The carrier may be a parenteral carrier, more preferably a solution that is isotonic with the blood of the recipient.

Examples of binders include, but are not limited to, microcrystalline cellulose and cellulose derivatives, gum tragacanth, glucose solution, acacia mucilage, gelatin solution, molasses, polvinylpyrrolidine, povidone, crospovidones, sucrose and starch paste.

Examples of diluents include salt.

Examples of excipients include, but are not limited to, surfactants, lipophilic vehicles, hydrophobic vehicles, sodium citrate, calcium carbonate, and dicalcium phosphate.

Examples of wetting agents include, but are not limited to, propylene glycol monostearate, sorbitan monooleate, diethylene glycol monolaurate and polyoxyethylene laural ether.

The artisan of ordinary skill in the art will recognize that many different ingredients can be used in formulations according to the present invention, in addition to the active agents, while maintaining effectiveness of the formulations in treating cancer. The list provided herein is not exhaustive.

For parenteral administration, in one embodiment, the agents of the invention can be formulated generally by mixing it at the desired degree of purity, in a unit dosage injectable form (solution, suspension, or emulsion), with a pharmaceutically acceptable carrier(s) described above.

Any dosage form used for therapeutic administration should be sterile. Sterility can readily be accomplished by filtration through sterile filtration membranes. Therapeutics generally are placed into a container having a sterile access port, for example, an intravenous solution bag or vial having a stopper pierceable by a hypodermic injection needle.

Kits of the Invention

According to another aspect of the invention, kits are provided. Kits according to the invention include package(s) comprising composition of the invention.

The phrase “package” means any vessel containing compositions presented herein. In preferred embodiments, the package can be a box or wrapping. Packaging materials for use in packaging pharmaceutical products are well known to those of skill in the art. Examples of pharmaceutical packaging materials include, but are not limited to, blister packs, bottles, tubes, inhalers, pumps, bags, vials, containers, syringes (including pre-filled syringes), bottles, and any packaging material suitable for a selected formulation and intended mode of administration and treatment.

The kit can also contain items that are not contained within the package but are attached to the outside of the package, for example, pipettes.

Kits may optionally contain instructions for administering compositions of the present invention to a subject having a condition in need of treatment. Kits may also comprise instructions for approved uses of components of the composition herein by regulatory agencies, such as the United States Food and Drug Administration. Kits may optionally contain labeling or product inserts for the present compositions. The package(s) and/or any product insert(s) may themselves be approved by regulatory agencies. The kits can include compositions in the solid phase or in a liquid phase (such as buffers provided) in a package. The kits also can include buffers for preparing solutions for conducting the methods, and pipettes for transferring liquids from one container to another.

The kit may optionally also contain one or more other compositions for use in combination therapies as described herein. In certain embodiments, the package(s) is a container for intravenous administration. In other embodiments, compositions are provided in an inhaler. In still other embodiments compositions are provided in a polymeric matrix or in the form of a liposome.

Methods of the Invention

The invention provides for a method for treating, inhibiting, or preventing the progression of a solid tumor cancer, in a subject. The method comprises administering to the subject, in need thereof, an effective amount of VLP vaccine or pharmaceutical composition of the invention so as to inhibit tumor growth or metastasis, kill tumor cells or reduce tumor burden. In another embodiment, the invention provides for a method of inhibiting tumor cells for a solid tumor which comprises contacting the tumor cells with an effective amount of a vaccine or composition of the invention.

The VLP vaccine or composition may be administered, e.g., in the case for cancer, by directly injection into or near a solid tumor. In a preferred embodiment, the administration may be intratumoral. Alternatively, in the case of cancer where a tumor site is not readily apparent, the administration may be made directly into or around the lymph node, spleen, thyroid, bone marrow, or other organ of the body with a high concentration of tumor cells. Further, depending on the site of the cancer, the administration may be intramuscular, intraperitoneal, intranasal, intradermal, or transmucosal.

In accordance with the practice of the invention, the VLP vaccine may be admixed with the therapeutic agent just prior to administration of the composition to the subject. Alternatively, the composition may be available premixed so as to contain both the VLP vaccine and the therapeutic agent.

The dosage may vary but includes a dose such that the total amount of CpG oligonucleotide is in the range of 0.001-0.05, 0.01-0.5, 0.1-5.0, 1-30, 20-60, 50-100, 90-300, or 250-1,000 milligrams per dose.

The solid tumor cancer may be an adrenal cancer, anal cancer, aplastic anemia, bile duct cancer, bladder cancer, bone cancer, brain/CNS cancer, breast cancer, cancer of unknown primary origin, Castleman Disease, cervical cancer, colon/rectum cancer, endometrial cancer, esophagus cancer, Ewing family of tumors, eye cancer, gallbladder cancer, gastrointestinal carcinoid tumors, Gastrointestinal Stromal Tumor (GIST), Gestational Trophoblastic Disease, Hodgkin Disease, Kaposi Sarcoma, Kidney Cancer, Laryngeal and Hypopharyngeal Cancer, Liver Cancer, Lung Cancer, Lymphoma, Malignant Mesothelioma, Multiple Myeloma, Myelodysplastic Syndrome, Nasal Cavity and Paranasal Sinus Cancer, Nasopharyngeal Cancer, Neuroblastoma, Non-Hodgkin Lymphoma, Oral Cavity and Oropharyngeal Cancer, Osteosarcoma, Ovarian Cancer, pancreatic cancer, Penile Cancer, Pituitary Tumors, prostate cancer, Retinoblastoma, Rhabdomyosarcoma, Salivary Gland Cancer, Sarcoma, Skin Cancer, Stomach Cancer, Testicular Cancer, Thymus Cancer, Thyroid Cancer, Uterine Sarcoma, Vaginal Cancer, Vulvar Cancer, Waldenstrom Macroglobulinemia, Wilms Tumor, non-Hodgkin lymphoma, Hodgkin lymphoma, Burkitt's lymphoma, lymphoblastic lymphomas, mantle cell lymphoma (MCL), multiple myeloma (MM), small lymphocytic lymphoma (SLL), splenic marginal zone lymphoma, marginal zone lymphoma (extra-nodal or nodal), mixed cell type diffuse aggressive lymphomas of adults, large cell type diffuse aggressive lymphomas of adults, large cell immunoblastic diffuse aggressive lymphomas of adults, small noncleaved cell diffuse aggressive lymphomas of adults, or follicular lymphoma.

In a further embodiment, the cancer may be any of head and neck cancer, breast, salivary gland, thyroid, pancreas, stomach, bladder, endometrial or uterine carcinoma, cervical cancer, ovarian, vulvar cancer, prostate, colon, rectal, colorectal, lung, non-small cell lung cancer, osteosarcoma, glioblastoma, kidney, liver, melanoma or metastatic cancer.

In one embodiment of the invention, the vaccine may enhance receptor signaling in the subject by having an ordered presentation of CpG oligonucleotide. In another embodiment, the vaccine may enhance receptor signaling (e.g., TLR9 receptor) in the subject by increasing cellular uptake of CpG oligonucleotide. The cells may be antigen presenting cells, lymphocytes, monocytes, or NK cells.

The invention also provides for a method for producing a VLP free of a viral genome protein comprising culturing the host vector system of the invention under suitable culture conditions so as to produce the VLP free of a viral genome in the host and recovering the VLP free of a viral genome so produced. Alternatively, the method comprises culturing the host vector system of the invention under suitable culture condition so as to produce VLP coat protein in the host, assembling VLP from VLP coat protein isolated from the host in the absence of a viral genome, and recovering the VLP free of a viral genome so produced. VLP may also be produced from assembly of VLP monomers following isolation from a host cell. For example, the VLP may be assembled from capsid proteins outside of the host cell. Alternatively, the VLP of the invention may be produced in a cell free in vitro transcription and/or translation system (Bundy 2008b, Bundy 2011).

In another example, the invention provides methods for producing, in a cell-free in vitro reaction, a VLP free of a viral genome. Preferably, the VLP is a population of icosahedral virus like particles free of a viral genome. This method may comprise synthesizing virus coat proteins in a prokaryotic cell-free in vitro translation reaction (e.g. substantially free of polyethylene glycol). The prokaryotic cell-free in vitro translation reaction may contain a bacterial cell extract, components of polypeptide and/or mRNA synthesis machinery; a template for transcription for the translation of the polypeptide; monomers for synthesis of the polypeptide; and co-factors, enzymes and other reagents necessary for translation to produce the virus coat proteins (e.g., at least about 250 ug/ml of the virus coat proteins) under conditions permissive for the virus coat proteins to self-assemble into a stable icosahedral virus like particle free of a viral genome which comprises at least 60 separate proteins.

In one embodiment, the VLP free of a viral genome is produced by the methods of the invention and may contain at least one unnatural amino acid (also referred to herein as non-natural amino acid or nnAA) used to conjugate it to a CpG oligonucleotide (supra.).

For attachment (also referred to herein as conjugation) of the CpG oligonucleotide to the VLP, the virus coat polypeptides of the VLP may be modified to comprise at least one first unnatural amino acid (also referred to herein as non-natural amino acid or non-canonical amino acid (nnAA)) at a site of interest, such as the incorporation of azidohomoalanine during virus coat polypeptide synthesis in the place of methionine, and the CpG oligonucleotide attached to an alkyne functional group, such as 5-octadiynyl dU at the 3′ end of the CpG oligonucleotide to produce CpG-X. The azide functional group of azidohomoalanine incorporated into a capsid protein of a VLP may participate in a (3+2) cycloaddition click reaction with an alkyne functional group of CpG-X, resulting in VLP crosslinked to CpG oligonucleotide. Other unnatural amino acid-containing capsid proteins within the same VLP may similarly participate in the (3+2) cycloaddition click reaction to produce a VLP attached or joined to a CpG oligonucleotide, producing a VLP with two or more CpG oligonucleotides. A similar strategy based on azide-alkyne functional group pairs may be used to attach a therapeutic agent or immune checkpoint inhibitor to VLP. While it is preferably to perform the (3+2) cycloaddition click reaction with azide and alkyne-containing reactants following formation of a VLP, such cycloaddition click reaction may be performed with VLP capsid protein or monomer before assembly into a VLP.

The following examples are provided to further illustrate aspects of the invention. These examples are non-limiting and should not be construed as limiting any aspect of the invention.

### EXAMPLES

### Example 1

Synthesis of HepB Core Protein with Azidohomoalanine as Methionine Replacement In Vivo and Purification of Assembled Azidohomoalanine-Containing HepB Core (HBC) VLPs

HepB core (HBC) protein with azidohomoalanine as methionine replacement is synthesized in vivo using a methionine (metB1) auxotroph, IPTG-inducible T7 RNA polymerase E. coli strain with HepB core coding sequences under the control of a T7 RNA phage promoter. The bacterial host strain is T7 Express Crystal Competent E. coli (High Efficiency; New England Biolabs) with methionine auxotroph E. coli mutation (metB1) and has the genotype of: fhuA2 lacZ::T7 gene1 [lon] ompT gal sulA11 R(mcr-73::miniTn10—Tet^{S})2 [dcm] R(zgb-210::Tn10—Tet⁵) endA1 metB1 Δ(mcrC-mrr)114::IS10. This bacterial strain is transformed with pLysS plasmid having a chloramphenicol resistance marker gene (CAM^{R}) and pET21-Hep B Core plasmid having an ampicillin resistance marker gene (Amp^{R}) and bearing a HepB core protein coding sequence under the control of a T7 RNA polymerase promoter. The HepB core coding sequence as provided in FIG. 1 (lower) is inserted between the Nde I and Sal I sites in the multiple cloning sequence (MCS) of the pET21a plasmid DNA (Novagen) to permit expression of the 149-amino acid HepB core protein (FIG. 1, upper). FIG. 5 shows a diagram of pET21-Hep B Core plasmid DNA (upper) and its sequence (lower). Selection condition for maintaining both pLysS and pET21-Hep B Core plasmids in the same bacterial cell is 100 μg/ml ampicillin and 35 μg/ml chloramphenicol.

Materials

M9 medium (100 mL) includes M9 salts (5×, Sigma) 20 mL; Glucose (20%; Sigma)* 2 mL; MgSO₄ (1 M; Fisher Scientific)** 200 μL; CaCl₂ (1 M; Fisher Scientific)** 10 μL; Amino Acid Mix (50×)* 2 mL; Vitamin B Complex (100×)* 1 mL; Ferric Ammonium Citrate (1 g/L) 200 μL; add Ampicillin (Sigma, Part# A9518) to 100 μg/ml; add Chloramphenicol (CalBiochem, Part#220551) to 35 μg/ml; and add H₂O to total 100 mL (* Filter-sterilized stocks stored at 4° C. and **Autoclaved stocks stored at room temperature).

Amino Acid Mix (50×) includes 1 g Arg (Sigma, Part# A3784); 1 g Glu (Sigma, Part# G5763); 1 g Lys (Sigma, Part# L5626); 1 g His (Sigma, Part# H8000); 1 g Gly (Sigma, Part# G7126); 1 g Ile (Sigma, Part# 112752); 1 g Phe (Sigma, Part# P2126); 1 g Leu (Sigma, Part# L8000); 1 g Cys (Sigma, Part# C8152); 1 g Asp (Sigma, Part# A4534); 1.5 g L-Val (Sigma, Part# V0500); 4 g L-Ser (Sigma, Part# S5511); 4 g L-Thr (Sigma, Part# T8625); and add H₂O to 200 mL.

Vitamin B Complex (100×) includes 100 mg riboflavin (Sigma, Part# R7649); 100 mg niacinamide (Sigma, Part# N5535); 100 mg pyridoxic HCl (Sigma, Part# P4722); 100 mg thiamine (Sigma, Part# T1270); 100 mg Biotin (Sigma, Part# B3010); and add H₂O to 100 mL.

Azidohomoalanine (AHA) Stock (from MedChem Source LLP or ACME Bioscience Inc.) includes 4 mg/mL (stored in −80° C. without light exposure).

VLP Re-suspension Buffer (1×) includes 50 mM Tris pH7.5 and 500 mM NaCl.

The following reagents were also used: LB medium (Sigma, Part# L3022); IPTG (Sigma, Part# 16758); PBS (Corning, Part# 21-040-CV); and saturated ammonium sulfate (Sigma, Part# A4418).

Induction of HepB Core Protein Containing Azidohomoalanine

T7 Express Crystal Competent E. coli (High Efficiency; New England Biolabs) transformed with both pLysS and pET21-Hep B Core plasmids are grown overnight in 2 mL of LB medium (with 100 μg/ml ampicillin and 35 μg/ml chloramphenicol) at 37° C. The next day, cells are diluted 100-fold into 10 mL of fresh LB medium (supplemented with ampicillin and chloramphenicol) and grown to log phase until OD600 of 0.5 at 37° C. at which point the cells are harvested by spinning at 1,000×g for 15 minutes. Supernatant is removed and cell pellet is resuspended in 1 mL of M9 medium. The cells are grown in M9 medium for 3 hours at 37° C., after which both IPTG (1 mM final concentration) and azidohomoalanine (AHA; 200 μg/ml final concentration) are added to induce expression of HepB core protein and allow incorporation of AHA in place of methionine. With the introduction of AHA into culture medium, the cells are grown in the dark by covering the culture flask to avoid light. After overnight culturing at 37° C., the cells are harvested by spinning at 1,000×g for 15 minutes. Supernatant is discarded and cell pellet is stored at −80° C.

Analysis of Induced Cells for HepB Core Protein Expression

The cell pellet is resuspended in 1 mL of PBS, and cells are sonicated for 15 seconds, three times. Soluble and insoluble components of the disrupted cells are separated from each other by centrifugation at 15,000×g for 15 minutes. The soluble component (supernatant) is collected and subjected to further purification to obtain isolated HepB core protein (below). The supernatant is also analyzed by SDS-PAGE following reduction of all disulfide bonds. HepB core monomer appears as a distinct band at 16 kDa.

Isolation of Azidohomoalanine-Containing HepB Core Protein (HBC-Azide) from Disrupted Cells

HepB core protein in the supernatant (from above, after sonication and centrifugation) is precipitated with ammonium sulfate by adding saturated ammonium sulfate drop-wise to the supernatant to a final 30% saturation, mixing for an additional hour, and centrifuging to pellet the precipitate. After removing supernatant, the ammonium sulfate precipitate of HepB core protein is resuspended in 1 mL of 1× PBS.

Formation of HBC VLP-Azide and Purification by Ammonium Sulfate Precipitation

To form VLP from HepB core protein with azidohomoalanine, the HepB core protein in PBS is dialyzed against 50-200 volumes of 0.5M NaCl pH 7.5. Following self-assembly of AHA-containing HepB core proteins, the resulting HBC VLP-azide particles are purified by two rounds of ammonium sulfate precipitation. Briefly, the dialyzed HBC VLP-azide particles are precipitated with 30% ammonium sulfate by adding 0.6 ml of saturated ammonium sulfate drop-wise into 1.4 ml solution containing dialyzed HBC VLP-azide particles and additional PBS to reach desired volume, followed by incubation for 1 hr at room temperature. The ammonium sulfate precipitate is spun at 14K for 10 min, and then the pellet is resuspended in 8 ml of PBS and further incubated for 1 hr at room temperature. A second round of ammonium sulfate precipitation is performed by adding saturated ammonium sulfate drop-wise to 30% and incubating for 1 hr at room temperature, followed by centrifugation at 14K for 10 min. The precipitate is resuspended in 1.4 ml PBS. The resuspended HBC VLP-azide particles are incubated for 1 hr at room temperature. Any insoluble material is removed by centrifugation at 14K for 10 min. The resulting supernatant is moved to a fresh 15 ml conical tube and HBC VLP-azide particles are purified by ammonium sulfate precipitation.

The concentration of the HBC VLP-azide particles is determined by absorbance at OD₂₈₀ with 1 mg/ml=1.77 OD₂₆₀. Purity of the HBC VLP-azide preparation is analyzed by SDS-PAGE.

Endotoxin Removal

The ammonium sulfate-purified HBC VLP-azide particles in PBS (from above) are chilled to 4° C. and 1/10^{th} volume of chilled 10% Triton™ X-114 is added. The solution is incubated at room temperature for 1 hr with frequent mixing, followed by centrifugation at 3,000×g to form a boundary. Aqueous (upper) layer is removed carefully. Additional aqueous layer near the boundary is removed to 0.5 ml tube and spun at 14K for 5 min to form a boundary. The upper layer is removed and added to the larger aqueous sample (1^{st} aqueous layer removed from the 1^{st} extraction). The Triton™ X-114 extraction procedure is repeated three additional times to further remove endotoxins.

To remove insoluble aggregates, the aqueous solution is incubated at room temperature for 1 hr and then centrifuged at 15,000×g for 15 minutes. The supernatant is collected taking care not to disturb the pellet. The supernatant is used to determine the protein concentration and is analyzed by reducing SDS-PAGE.

Further Purification of HBC VLP-azide

The isolated HBC VLP-azide preparation, following successive ammonium sulfate precipitations and endodoxin removal protocol, may be further purified by affinity chromatography, immunoaffinity chromatography, size exclusion chromatography, velocity sedimentation, equilibrium sedimentation, immunoprecipitation, dialysis, filtration, electrophoretic methods, and/or differential precipitation.

In general when exchanging buffer into PBS, a centrifugal filter unit (Millipore, Part #UFC510024) is used for sample volumes <1 ml; whereas, PD-10 desalting column (GE Healthcare, Part #17-0851-01) is used for sample volumes of 2-10 ml.

HBC VLP-azides are stored at −80° C., −20° C. or 4° C.

Conjugation of CpG Oligonucleotide to VLP

A CpG-containing oligonucleotide with a cross-linkable functional group (CpG-X) was synthesized and purified by Sigma Custom Oligo Unit (http://www.sigmaaldrich.com/life-science/custom-oligos.html). The sequence used is 5′ TsGsAsCsTsGsTsGsAsAsCGsTsTsCsGsAsGsAsTsGsA-{5-Oct-dU} 3′, where ‘s’ denotes a phosphorothioate linkage in the sequence and 5-Oct-dU is 5-octadiynyl dU at the 3′ end of the oligonucleotide. Presence of a 5-Oct-dU moiety introduces an alkyne functional group to the CpG oligonucleotide, and the resulting CpG-X oligonucleotide is also referred to as CpG-alkyne. 5 octadiynyl dU attached at the 3′ end of the oligo formed the basis of alkyne-azide conjugation to the VLP.

HBC VLP-azide was mixed with CpG-alkyne, sodium ascorbate, Tween-20 and phosphate buffered saline in an opaque reaction chamber. The mixture was overlayed with argon gas. The catalyst, tetrakis(acetonitrile)copper(I)hexafluorophosphate [tetrakis Cu(I), Sigma], and enhancer, tris(triazoylmethyl)amine [TTMA, Shanghai ChemPartner] were then added and the reaction was allowed to proceed overnight at room temperature with mild agitation. Final reaction conditions were as follows when complete saturation of the VLP with CpG oligonucleotide was desired: HBC VLP-azide 60 p,M; CpG-alkyne 80 p,M; Na ascorbate 200 μM; Tween .01%; 10 mM potassium phosphate, at pH 8; TTMA 0.25 mM; Tetrakis Cu(I) 500 μM; 30° C., overnight, dark.

Analysis of CpG Oligonucleotide Conjugation

SDS-PAGE

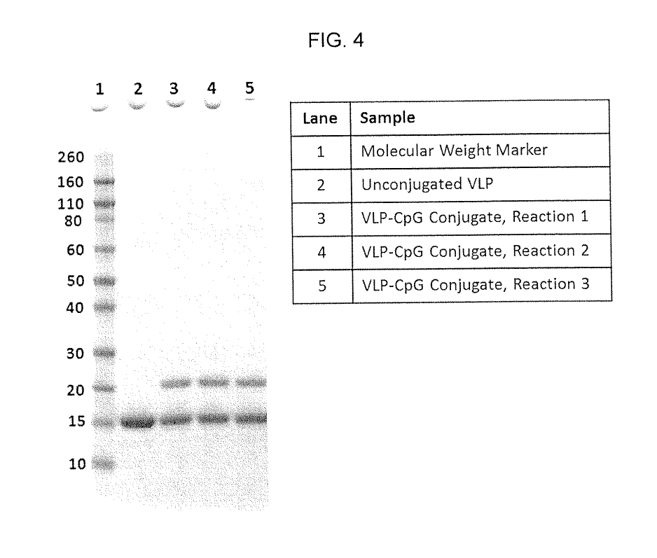

VLP-CpG oligonucleotide conjugation was assessed by observing gel mobility shifts of the HBC monomer on reducing SDS-PAGE gels as shown in FIG. 4.

### Example 2

Intratumoral Injection of CpG Oligonucleotide-Bearing Virus-Like Particles for Treatment of Triple Negative Breast Tumors in Mice

Materials

The following CpG oligonucleotides is used: (1) CpG with sequence 5′-tccatgacgttectgacgtt-3′ (lowercase indicates phosphorothioate bonds) as control; (2) CpG-alkyne (5′-tgactgtgaaCGttcgagatga-5 octadiynyl dU-3′); and (3) CpG-VLP. 4T1 tumor cells ((ATCC CRL-2539), derived from mouse and used in animal model of stage IV human breast cancer, will be injected in the animals.

Animal Care

Female Balb/c mice (6-8 weeks old) is obtained from Charles River Labs. Animals are housed in the animal facility under an approved IACUC protocol. Tumor cell injections, caliper measurements of tumors, injection of therapeutics, animal euthanasia and tumor tissue harvesting are performed.

In Vivo Tumor Model and Therapy

4T1, an aggressive, triple-negative mammary carcinoma is implanted bilaterally into syngeneic BALB/C mice. Treatment of one of the bilateral tumors will be initiated when tumors are 80-100 mm³ (clay 9-11). Five groups of 12 mice each will be treated as described in the following table:

| | | | CpG | |
|---|---|---|---|---|
| | | | oligonucleotide/ | |
| Group | Description | Mice | dose (ug) | Doses |
| 1 | PBS Control | 12 | | 3 |
| 2 | CpG control | 12 | 50 | 3 |
| 3 | CpG -3′ 5 Oct dU | 12 | 50 | 3 |
| 4 | CpG -3′ 5 Oct dU | 12 | 10 | 3 |
| 5 | VLP-CpG | 12 | 10 | 3 |

In each case, the therapeutic agent is injected intra-tumorally 3 times on alternate days over a 5 day period. Mice are sacrificed if tumors become ulcerated or reach a diameter >2 cm. To study the effects of treatment on leukocyte infiltrates within the tumor, 3 mice from each group are euthanized 3-4 days after the last injection of therapeutic agents and tumors harvested for analyses. The study ends on day 30-35 after the start of treatment and all surviving animals are euthanized and tissues/tumors are harvested for analysis.

Analyses

Three to 4 days after the final injection of therapeutic agents, 3 mice from each group are euthanized for immunological analyses. Serum or plasma is collected and stored frozen at −80° C. for future assays (e.g., for serum cytokine assays, immunofingerprinting of antibody reactivities). Tumors are harvested and fixed in formalin for subsequent analyses of infiltrating cells (T cells, Treg cells, macrophages, dendritic cells, B cells, NK cells, and myeloid derived suppressor cells (MDSCs)).

In the remaining 9 animals per group, growth of the treated tumors and the untreated contralateral tumors are assessed twice per week via caliper measurements. Tumor volumes are calculated as: [(tumor length)×(tumor width)²×(π/6)]. General animal health are also monitored, e.g., activity, weight, coat appearance.

At the end of the study, mice are euthanized and tumors are harvested and weighed. Lungs are also removed, weighed, and observed for metastatic tumor nodules.

### REFERENCES

1. Siano, M., et al., A phase I-II study to determine the maximum tolerated infusion rate of rituximab with special emphasis on monitoring the effect of rituximab on cardiac function. Clin Cancer Res, 2008. 14(23): p. 7935-9.
2. Witzig, T. E., et al., Rituximab therapy for patients with newly diagnosed, advanced-stage, follicular grade I non-Hodgkin's lymphoma: a phase II trial in the North Central Cancer Treatment Group. J Clin Oncol, 2005. 23(6): p. 1103-8.
3. Spina, M., et al., Rituximab plus infusional cyclophosphamide, doxorubicin, and etoposide in HIV-associated non-Hodgkin lymphoma: pooled results from 3 phase 2 trials. Blood, 2005. 105(5): p. 1891-7.
4. Hainsworth, J. D., et al., Rituximab plus short-duration chemotherapy as first-line treatment for follicular non-Hodgkin's lymphoma: a phase II trial of the minnie pearl cancer research network. J Clin Oncol, 2005. 23(7): p. 1500-6.
5. Bendandi, M., Idiotype vaccines for lymphoma: proof-of-principles and clinical trial failures. Nat Rev Cancer, 2009. 9(9): p. 675-81.
6. Kwak, L. W., et al., Induction of immune responses in patients with B-cell lymphoma against the surface-immunoglobulin idiotype expressed by their tumors. N Engl J Med, 1992. 327(17): p. 1209-15.
7. Miller, R. A., et al., Treatment of B-cell lymphoma with monoclonal anti-idiotype antibody. N Engl J Med, 1982. 306(9): p. 517-22.
8. Schuster, S. J., et al. Idiotype vaccine therapy (BiovaxID) in follicular lymphoma in first complete remission: BV301 Phase III clinical trial results. in American Society of Clinical Oncology. 2009.
9. Ai, W. Z., et al., Anti-idiotype antibody response after vaccination correlates with better overall survival in follicular lymphoma. Blood, 2009. 113(23): p. 5743-6.
10. McCormick, A. A., et al., Plant-produced idiotype vaccines for the treatment of non-Hodgkin's lymphoma: safety and immunogenicity in a phase I clinical study. Proc Natl Acad Sci USA, 2008. 105(29): p. 10131-6.
11. Milner, K., et al. Development of quantitative methods to assess humoral immune response (IR) in follicular Non-Hodgkin's Lymphoma (fNHL) patients receiving idiotype-keyhole limpet hemocyanin (Id-KLH) active immunotherapy. in American Association of Cancer Reserach Annual Meeting. 2007. Poster #1857.
12. Hennessy, E. J., A. E. Parker, and L. A. O'Neill, Targeting Toll-like receptors: emerging therapeutics? Nat Rev Drug Discov, 2010. 9(4): p. 293-307.
13. Krieg, A. M., Toll-like receptor 9 (TLR9) agonists in the treatment of cancer. Oncogene, 2008. 27(2): p. 161-7.
14. Murata, M., Activation of Toll-like receptor 2 by a novel preparation of cell wall skeleton from Mycobacterium bovis BCG Tokyo (SMP-105) sufficiently enhances immune responses against tumors. Cancer Sci, 2008. 99(7): p. 1435-40.
15. Mizel, S. B. and J. T. Bates, Flagellin as an adjuvant: cellular mechanisms and potential. J Immunol. 185(10): p. 5677-82.
16. Basith, S., et al., Toll-like receptor modulators: a patent review (2006-2010). Expert Opin Ther Pat, 2011. 21(6): p. 927-44.
17. Bundy, B. C. and J. R. Swartz, Efficient disulfide bond formation in virus-like particles. J Biotechnol, 2011. 154(4): p. 230-9.
18. Bundy, B. C. and J. R. Swartz, Site-specific incorporation of p-propargyloxyphenylalanine in a cell-free environment for direct protein-protein click conjugation. Bioconjug Chem, 2010. 21(2): p. 255-63.
19. Bundy, B. C., M. J. Franciszkowicz, and J. R. Swartz, Escherichia coli-based cell-free synthesis of virus-like particles. Biotechnol Bioeng, 2008. 100(1): p. 28-37.
20. Kanter, G., et al., Cell-free production of scFv fusion proteins: an efficient approach for personalized lymphoma vaccines. Blood, 2007. 109(8): p. 3393-9.
21. Voloshin, A. M. and J. R. Swartz, Efficient and scalable method for scaling up cell free protein synthesis in batch mode. Biotechnol Bioeng, 2005. 91(4): p. 516-21.
22. Yang, J., et al., Expression of active murine granulocyte-macrophage colony-stimulating factor in an Escherichia coli cell-free system. Biotechnol Prog, 2004. 20(6): p. 1689-96.
23. Zlotnick, A., et al., Dimorphism of hepatitis B virus capsids is strongly influenced by the C-terminus of the capsid protein. Biochemistry, 1996. 35(23): p. 7412-21.
24. Pumpens, P. and E. Grens, HBV core particles as a carrier for B cell/T cell epitopes. Intervirology, 2001. 44(2-3): p. 98-114.
25. Patel, K. G. and J. R. Swartz, Surface functionalization of virus-like particles by direct conjugation using azide-alkyne click chemistry. Bioconjug Chem, 2011. 22(3): p. 376-87.
26. Goerke, A. R. and J. R. Swartz, High-level cell-free synthesis yields of proteins containing site-specific non-natural amino acids. Biotechnol Bioeng, 2009. 102(2): p. 400-16.
27. Patel, K. G., et al., Escherichia coli-based production of a tumor idiotype antibody fragment—tetanus toxin fragment C fusion protein vaccine for B cell lymphoma. Protein Expr Purif, 2010. 75(1): p. 15-20.
28. Zhou, Z. and C. J. Fahrni, A fluorogenic probe for the copper(I)-catalyzed azide-alkyne ligation reaction: modulation of the fluorescence emission via 3 (n,pi)-1 (pi,pi) inversion. J Am Chem Soc, 2004. 126(29): p. 8862-3.
29. Marshall, J. D. et al., Identification of a novel CpG DNA class and motif that optimally stimulate B cell and plasmacytoid dendritic cell functions. J. Leukoc. Biol. 2003. 73:781-92
30. Hartmann, G. et al., Rational design of new CpG oligonucleotides that combine B cell activation with high IFN-a indication in plasmacytoid dendritic cells. Eur. J. Immunol. 2003. 33:1633-41.

1. (canceled)

2. A pharmaceutical composition for treatment of cancer comprising (1) a vaccine comprising (a) a VLP attached to a CpG oligonucleotide and (b) one or more non-toxic pharmaceutically acceptable carrier or diluent, wherein the VLP attached to the CpG oligonucleotide is the only active ingredient in the vaccine and (2) a therapeutic agent admixed therewith.

3. The pharmaceutical composition of claim 2, wherein the VLP comprises virus coat polypeptides modified to comprise at least one first unnatural amino acid at a site of interest and wherein the CpG oligonucleotide is modified to comprise a reactive functional group which participates in a chemical reaction with the first unnatural amino acid to form a covalent bond.

4. The pharmaceutical composition of claim 2, wherein the CpG oligonucleotide attached to a VLP protein monomer is in an amount (molar) such that the CpCi oligonucleotide to VLP monomer ratio is equivalent to 1:24 to 1:12, 1:12 to 1:6, 1:6 to 1:3, 1:3 to 2:3 or 1:2 to 1:1.

5. The pharmaceutical composition of claim 2, wherein the CpG oligonucleotide is attached to the VLP in an average amount equivalent to 10 to 50 copies per VLP, 40 to 80 copies per VLP, 70 to 170 copies per VLP, or 160 to 240 copies per VLP.

6. The pharmaceutical composition of claim 2, wherein the CpG oligonucleotide comprises a sequence, 5′ TGACTGTGAACGTTCGAGATGA-3′(SEQ ID NO: 49).

7. The pharmaceutical composition of claim 6, wherein the sequence has a mixture of phosphodiester and phosphorothioate bonds as shown in 5′ T*G*A*C*T*G*T*G*A*A*CG*T*T*C*G*A*G*A*T*G*A 3′ (SEQ ID NO: 11), where * represents replacement of a phosphodiester bond with a phosphorothioate bond.

8. The pharmaceutical composition of claim 6, wherein the sequence has phosphorothioate bonds as shown in 5′ T*G*A*C*T*G*T*G*A*A*C*G*T*T*C*G*A*G*A*T*G*A 31SEQ ID NO: 13), where * represents replacement of a phosphodiester bond with a phosphorothioate bond.

9. The pharmaceutical composition of claim 6, wherein the CpG oligonucleotide further comprises a 5-octadiynyl deoxyuridine or a modified deoxyuridine or a linker at the 3′ end of the sequence.

10. The pharmaceutical composition of claim 2, wherein the VLP is formed by a hepatitis B core protein which comprises a sequence as shown in FIG. 1 or 5 or a portion thereof.

11. The pharmaceutical composition of claim 2, wherein the VLP comprises virus coat proteins or portions thereof from a virus selected from the group consisting of a bacteriophage, adenovirus, coxsackievirus, Hepatitis A virus, poliovirus, Rhinovirus, Herpes simplex virus, Varicella-zoster virus, Epstein-Barr virus, Human cytomegalovirus, Human herpes virus, Hepatitis B virus, Hepatitis C virus, yellow fever virus, dengue virus, West Nile virus, HIV, Influenza virus, Measles virus, Mumps virus, Parainfluenza virus, Respiratory syncytial virus, Human metapneumovirus, Human papillomavirus, Rabies virus, Rubella virus, Human bocarivus or Parvovirus, and Norovirus.

12. The pharmaceutical composition of claim 2, wherein the therapeutic agent is an immune checkpoint inhibitor.

13. The pharmaceutical composition of claim 12, wherein the immune checkpoint inhibitor is selected from a group consisting of a PD-1 inhibitor, a PDL1 inhibitor, a PDL2 inhibitor, a B7-H3 inhibitor, a B7-H4 inhibitor, a CTLA-4 inhibitor, a LAG3 inhibitor, a KIR inhibitor, a TIM3 inhibitor, a TIGIT inhibitor, a BTLA inhibitor, a CD160 inhibitor, an A2aR inhibitor, and a VISTA inhibitor.

14. The pharmaceutical composition of claim 12, wherein the immune checkpoint inhibitor is an antibody or fragment or derivative thereof that blocks activity of an immune checkpoint protein.

15. The pharmaceutical composition of claim 2, wherein the therapeutic agent is an anti-cancer agent selected from the group consisting of lenalidomide; ipilimumab; rituximab; alemtuzumab; ofatumumab; flavopiridol; Adriamycin; Dactinomycin; Bleomycin; Vinblastine; Cisplatin; ABT-199; acivicin; aclarubicin; acodazole hydrochloride; acronine; adozelesin; aldesleukin; altretamine; ambomycin; ametantrone acetate; amino glutethimide; amsacrine; anastrozole; anthramycin; asparaginase; asperlin; azacitidine; azetepa; azotomycin; batimastat; benzodepa; bicalutamide; bisantrene hydrochloride; bizelesin; bleomycin sulfate; brequinar sodium; bropirimine; busulfan; cactinomycin; calusterone; caracemide; carbetimer; carboplatin; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexonnaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; ibrutinib, idelalisib, idarubicin hydrochloride; ifosfamide; ilmofosine; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; obinutuzumab; ormaplatin; oxisuran; pegaspargase; peliomycin; pentamustine; peplomycin sulfate; perfosfamide; pipobroman; piposulfan; piroxantrone hydrochloride; plicamycin; plomestane; porfmer sodium; porfiromycin; prednimustine; procarbazine hydrochloride; puromycin; puromycin hydrochloride; pyrazofurin; riboprine; rituximab; rogletimide; safingol; safingol hydrochloride; semustine; simtrazene; sparfosate sodium; sparsomycin; spirogerranium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfm; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

16. (canceled)

17. (canceled)

18. (canceled)

19. (canceled)

20. (canceled)

21. (canceled)

22. (canceled)

23. (canceled)

24. (canceled)

25. (canceled)

26. A method of treating a subject with a cancer, comprising administering an effective amount of a pharmaceutical composition for treatment of cancer comprising (1) a vaccine comprising la) a VLP attached to a CpG oligonucleotide and (b) one or more non-toxic pharmaceutically acceptable carrier or diluent, wherein the VLP attached to the CpG oligonucleotide is the only active ingredient in the vaccine and (2) a therapeutic agent admixed therewith to the subject so as to inhibit the cancer thereby treating the subject.

27. (canceled)

28. A method of inhibiting tumor cells which comprises contacting the tumor cells with an effective amount of a pharmaceutical composition for treatment of cancer comprising (1) a vaccine comprising (a) a VLP attached to a CpG oligonucleotide and (b) one or more non-toxic pharmaceutically acceptable carrier or diluent, wherein the VLP attached to the CpG oligonucleotide is the only active ingredient in the vaccine and (2) a therapeutic agent admixed therewith thereby inhibiting the tumor cells.

29. (canceled)

30. The method of claim 26, wherein the subject is a mammal.

31. The method of claim 30, wherein the mammal is any of a human, monkey, ape, dog, cat, cow, horse, rabbit, mouse, or rat.

32. The method of claim 26, wherein the cancer comprises breast cancer, colon cancer, pancreatic cancer, prostate cancer, lung cancer, non-Hodgkin lymphoma, Hodgkin lymphoma, Burkitt's lymphoma, lymphoblastic lymphomas, mantle cell lymphoma is (MCL), multiple myeloma (MM), small lymphocytic lymphoma (SLL), head and neck cancer, melanoma or follicular lymphoma.

33. (canceled)

34. A method of inhibiting the growth of a solid tumor comprising intratumorally administering to a subject the composition of claim 2 in an amount effective to inhibit growth of the solid tumor.

35. (canceled)


SEQUENCE LISTING (49 sequences)

SEQ ID NO: 1 - DNA, 447 bp, Hepatitis B virus
atggacattgatccttacaaagagttcggcgcgaccgtagaacttctttcgtttctgccgagcgattttttcccgtcagttcgtgatctgctggacactgctgccgcactgtaccgtgatgctctcgagagcccggaacactgcagcccgcatcataccgcgctgcgtcaggcaatcctgtgctggggtgatttatccacgctggccacctgggtgggtacgaacatggaagatccggcttcgcgcgatctggttgtctcttatgtcaacaccaatgtgggcttgaagttccgtcagttgctttggtttcacatcagctgcctgacctttggacgcgaaactgtgctggaatatctggtgagctttggtgtctggatccgcaccccacctgcttatcgcccgcccaacgcgccgattctttcaacgctcccggaaactactgtggtg

SEQ ID NO: 2 - protein, 149 aa, Hepatitis B virus
MDIDPYKEFGATVELLSFLPSDFFPSVRDLLDTAAALYRDALESPEHCSPHHTALRQAILCWGDLSTLATWVGTNMEDPASRDLVVSYVNTNVGLKFRQLLWFHISCLTFGRETVLEYLVSFGVWIRTPPAYRPPNAPILSTLPETTVV

SEQ ID NO: 3 - DNA, 23 bp, Artificial Sequence
  CpG-X
tgactgtgaacgttcgagatgan

SEQ ID NO: 4 - DNA, 23 bp, Artificial Sequence
  CpG-X
tgactgtgaacgttcgagatgan

SEQ ID NO: 5 - DNA, 23 bp, Artificial Sequence
  CpG-X
tgactgtgaacgttcgagatgan

SEQ ID NO: 6 - DNA, 23 bp, Artificial Sequence
  CpG-X
tgactgtgaacgttcgagatgan

SEQ ID NO: 7 - DNA, 23 bp, Artificial Sequence
  CpG-X
tgactgtgaacgttcgagatgan

SEQ ID NO: 8 - DNA, 23 bp, Artificial Sequence
  CpG-X
ntgactgtgaacgttcgagatga

SEQ ID NO: 9 - DNA, 23 bp, Artificial Sequence
  CpG-X
tgactgtgaacgttcgagatgan

SEQ ID NO: 10 - DNA, 23 bp, Artificial Sequence
  CpG-X
ntgactgtgaacgttcgagatga

SEQ ID NO: 11 - DNA, 23 bp, Artificial Sequence
  CpG-X
tgactgtgaacgttcgagatgan

SEQ ID NO: 12 - DNA, 23 bp, Artificial Sequence
  CpG-X
ntgactgtgaacgttcgagatga

SEQ ID NO: 13 - DNA, 23 bp, Artificial Sequence
  CpG-X
tgactgtgaacgttcgagatgan

SEQ ID NO: 14 - DNA, 23 bp, Artificial Sequence
  CpG-X
ntgactgtgaacgttcgagatga

SEQ ID NO: 15 - DNA, 5846 bp, Artificial Sequence
  pET21-Hep B Core 319.44.1.4 Plasmid Sequence
taatacgactcactataggggaattgtgagcggataacaattcccctctagaaataattttgtttaactttaagaaggaggtatacatatggacattgatccttacaaagagttcggcgcgaccgtagaacttctttcgtttctgccgagcgattttttcccgtcagttcgtgatctgctggacactgctgccgcactgtaccgtgatgctctcgagagcccggaacactgcagcccgcatcataccgcgctgcgtcaggcaatcctgtgctggggtgatttatccacgctggccacctgggtgggtacgaacatggaagatccggcttcgcgcgatctggttgtctcttatgtcaacaccaatgtgggcttgaagttccgtcagttgctttggtttcacatcagctgcctgacctttggacgcgaaactgtgctggaatatctggtgagctttggtgtctggatccgcaccccacctgcttatcgcccgcccaacgcgccgattctttcaacgctcccggaaactactgtggtgtaatgaagcttgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaat

SEQ ID NO: 16 - DNA, 27 bp, Artificial Sequence
  Synthetic CpG oligonucleotide sequence designated M353
tcgtcgttgtcgaacgttcgagatgat

SEQ ID NO: 17 - DNA, 21 bp, Artificial Sequence
  Synthetic CpG oligonucleotide sequence designated M355
tcgtcgttcgaacgagatgat

SEQ ID NO: 18 - DNA, 29 bp, Artificial Sequence
  Synthetic CpG oligonucleotide sequence designated M354
tcgtcgttttgtcgaacgtccgagatgat

SEQ ID NO: 19 - DNA, 21 bp, Artificial Sequence
  Synthetic CpG oligonucleotide sequence designated M352
tcgtcgaacgttcgagatgat

SEQ ID NO: 20 - DNA, 21 bp, Artificial Sequence
  Synthetic CpG oligonucleotide sequence designated C593
tcgtcgagcgctcgagatgat

SEQ ID NO: 21 - DNA, 21 bp, Artificial Sequence
  Synthetic CpG oligonucleotide sequence designated C594
tcgtcgatcgatcgagatgat

SEQ ID NO: 22 - DNA, 21 bp, Artificial Sequence
  Synthetic CpG oligonucleotide sequence designated C595
tcgtcggtcgaccgagatgat

SEQ ID NO: 23 - DNA, 21 bp, Artificial Sequence
  Synthetic CpG oligonucleotide sequence designated C546
tcgtcgttcgaacgagatgat

SEQ ID NO: 24 - DNA, 21 bp, Artificial Sequence
  Synthetic CpG oligonucleotide sequence designated C640
tcgtcgggcgcccgagatgat

SEQ ID NO: 25 - DNA, 21 bp, Artificial Sequence
  Synthetic CpG oligonucleotide sequence designated C642
tcgtcgcgcgcgcgagatgat

SEQ ID NO: 26 - DNA, 21 bp, Artificial Sequence
  Synthetic CpG oligonucleotide sequence designated C644
tcgtcgcccgggcgagatgat

SEQ ID NO: 27 - DNA, 21 bp, Artificial Sequence
  Synthetic CpG oligonucleotide sequence designated M356
tcgtcgacgatcgtcgatgat

SEQ ID NO: 28 - DNA, 21 bp, Artificial Sequence
  Synthetic CpG oligonucleotide sequence designated M357
tcgtcgtcgtacgacgatgat

SEQ ID NO: 29 - DNA, 28 bp, Artificial Sequence
  Synthetic CpG oligonucleotide sequence designated M361
tcgtcgttgtcgttcgaacgacgttgat

SEQ ID NO: 30 - DNA, 25 bp, Artificial Sequence
  Synthetic CpG oligonucleotide sequence designated M362
tcgtcgtcgttcgaacgacgttgat

SEQ ID NO: 31 - DNA, 27 bp, Artificial Sequence
  Synthetic CpG oligonucleotide sequence designated M358
tcgtcgttctcgacgatcgtcgatgat

SEQ ID NO: 32 - DNA, 21 bp, Artificial Sequence
  Synthetic CpG oligonucleotide sequence designated 1018
tgactgtgaacgttcggatga

SEQ ID NO: 33 - DNA, 21 bp, Artificial Sequence
  Synthetic CpG oligonucleotide sequence designated C274
tcgtcgaacgttcgagatgat

SEQ ID NO: 34 - DNA, 22 bp, Artificial Sequence
  Synthetic CpG oligonucleotide sequence designated C583
tcgaacgttcgaacgttcgaat

SEQ ID NO: 35 - DNA, 23 bp, Artificial Sequence
  Synthetic CpG oligonucleotide sequence designated C582
ttcgaacgttcgaacgttcgaat

SEQ ID NO: 36 - DNA, 24 bp, Artificial Sequence
  Synthetic CpG oligonucleotide sequence designated C637
tcaacgttcgaacgttcgaacgtt

SEQ ID NO: 37 - DNA, 24 bp, Artificial Sequence
  Synthetic CpG oligonucleotide sequence designated 2006
tcgtcgttttgtcgttttgtcgtt

SEQ ID NO: 38 - DNA, 21 bp, Artificial Sequence
  Synthetic CpG oligonucleotide sequence designated C630
tcgacgtcgacgtccacgtat

SEQ ID NO: 39 - DNA, 21 bp, Artificial Sequence
  Synthetic CpG oligonucleotide sequence designated C631
tcgtcgaaacgtttcgacagt

SEQ ID NO: 40 - DNA, 22 bp, Artificial Sequence
  Synthetic CpG oligonucleotide sequence designated C633
tcgtcgaaaacgttttcgagat

SEQ ID NO: 41 - DNA, 20 bp, Artificial Sequence
  Synthetic CpG oligonucleotide sequence designated 2216
gggggacgatcgtcgggggg

SEQ ID NO: 42 - DNA, 24 bp, Artificial Sequence
  Synthetic CpG oligonucleotide sequence designated 2006
tcgtcgttttgtcgttttgtcgtt

SEQ ID NO: 43 - DNA, 25 bp, Artificial Sequence
  Synthetic CpG oligonucleotide sequence designated M383
tgctgctgcttgcaagcagcttgat

SEQ ID NO: 44 - DNA, 25 bp, Artificial Sequence
  Synthetic CpG oligonucleotide sequence designated M384
tcgtcgtcgttcgaacgacgttgat

SEQ ID NO: 45 - DNA, 25 bp, Artificial Sequence
  Synthetic CpG oligonucleotide sequence designated M385
tcgtcgtgcttgcaagcacgttgat

SEQ ID NO: 46 - DNA, 25 bp, Artificial Sequence
  Synthetic CpG oligonucleotide sequence designated M386
tcgtcgtcgatcgtacgacgttgat

SEQ ID NO: 47 - DNA, 20 bp, Artificial Sequence
  Synthetic CpG oligonucleotide sequence designated M387
tcgtcgtcgttcgaacgacg

SEQ ID NO: 48 - DNA, 25 bp, Artificial Sequence
  Synthetic CpG oligonucleotide sequence designated M388
tcgtcgtcgttcgaacgacgtcgtt

SEQ ID NO: 49 - DNA, 22 bp, Artificial Sequence
  Synthetic CpG oligonucleotide sequence
tgactgtgaacgttcgagatga